(19) United States
(12) Patent Application Publication
(10) Pub. No.: US 2023/0111253 A1
(43) Pub. Date: Apr. 13, 2023

(54) MICRONEEDLE ARRAY SENSOR PATCH FOR CONTINUOUS MULTI-ANALYTE DETECTION

(71) Applicant: THE REGENTS OF THE UNIVERSITY OF CALIFORNIA, Oakland, CA (US)

(72) Inventors: Joseph Wang, San Diego, CA (US); Farshad E. Tehrani, La Jolla, CA (US); Patrick Mercier, La Jolla, CA (US); Christopher Brown, La Jolla, CA (US); Hazhir Teymourian, La Jolla, CA (US)

(21) Appl. No.: 17/904,284

(22) PCT Filed: Feb. 22, 2021
(86) PCT No.: PCT/US2021/019107

§ 371 Date: Aug. 15, 2022

Related U.S. Application Data
(60) Provisional application No. 62/980,084, filed on Feb. 21, 2020.
(60) Provisional application No. 63/005,076, filed on Apr. 3, 2020.

(51) Int. Cl. A61B 5/145; A61B 5/1473; A61B 5/00

(52) U.S. Cl. CPC A61B 5/14546; A61B 5/002; A61B 5/685; A61B 5/742; A61B 5/6831; A61B 5/6833; A61B 5/14514; A61B 5/14735; A61B 2560/0209; A61B 2560/0214; A61B 2562/066; A61B 2562/164; A61B 2562/166; A61B 2562/182

(57) ABSTRACT

Disclosed are systems, devices and methods for continuous and simultaneous monitoring of multiple analytes within interstitial fluid by an integrated system for a microneedle array sensor platform. In some aspects, the device includes a microneedle array sensor unit, an electronics unit and a housing structure. The electronics unit is in electrical communication with an array of electrode probe structures via an array of surface-mount, spring loaded pins, and the electronics unit includes a power source, a data processing unit, and a wireless transmitter. The housing structure is configured to encase, at least partially, the microneedle array sensor unit and the electronics unit, where the array of microneedles is exposed from a side of the housing structure. The device can be configured as a patch worn on skin of a patient user.

### CROSS-REFERENCE TO RELATED APPLICATIONS

This patent document claims priorities to and benefits of U.S. Provisional Pat. Application No. 62/980,084, titled “MICRONEEDLE ARRAY SENSOR PATCH FOR CONTINUOUS MULTI-ANALYTE DETECTION” and filed on Feb. 21, 2020, and U.S. Provisional Pat. Application No. 63/005,076, titled “MICRONEEDLE ARRAY SENSOR PATCH FOR CONTINUOUS MULTI-ANALYTE DETECTION” and filed on Apr. 3, 2020. The entire content of the aforementioned patent applications is incorporated by reference as part of the disclosure of this patent document.

### TECHNICAL FIELD

This patent document relates to biosensor technology.

### BACKGROUND

Biosensors can provide real-time detection of physiological substances and processes in living things. A biosensor is an analytical tool that can detect a chemical, substance, or organism using a biologically sensitive component coupled with a transducing element to convert a detection event into a signal for processing and/or display. Biosensors can use biological materials as the biologically sensitive component, e.g., such as biomolecules including enzymes, antibodies, nucleic acids, etc., as well as living cells. For example, molecular biosensors can be configured to use specific chemical properties or molecular recognition mechanisms to identify target agents.

### SUMMARY

Disclosed are systems, devices and methods for continuous and simultaneous monitoring of multiple analytes within interstitial fluid by an integrated system for a microneedle array sensor platform.

In some aspects, disclosed are embodiments of a wearable microneedle medical device for simultaneous and continuous monitoring of a plurality of analytes in a fluid in a non-invasive fashion. In some embodiments, for example, a wearable microneedle medical device includes a microneedle array sensor unit, an electronics unit and a housing structure. The microneedle array sensor unit includes an array of microneedles, each comprising three or more exterior walls forming a protruding needle structure converging at an apex point, wherein each microneedle includes a hollow interior defined by an interior wall, and an opening to the hollow interior on at least one of the three or more exterior walls of the protruding needle structure, an array of electrode probe structures, each disposed within the hollow interior of a respective protruding needle structure, wherein a first electrode probe structure of the array is configured to interact with a first analyte that comes in contact with the first electrode probe structure via the opening to the hollow interior to produce a first electrical signal indicative of an electrochemical reaction involving the first analyte, and wherein a second electrode probe structure of the array is configured to interact with a second analyte that comes in contact with the second electrode probe structure via the opening to the hollow interior to produce a second electrical signal indicative of an electrochemical reaction involving the second analyte. The electronics unit is in electrical communication with the array of electrode probe structures, and includes a power source, a data processing unit, and a wireless transmitter. The housing structure is configured to encase, at least partially, the microneedle array sensor unit and the electronics unit, where the array of microneedles is exposed from a side of the housing structure. The device can be configured as a patch worn on skin of a patient user. Other example embodiments are described that may include additional or alternative features.

In some aspects, disclosed are embodiments of a wearable microneedle sensor device for detecting an analyte in a fluid via suction. In some embodiments, for example, a wearable microneedle sensor device includes a body that encloses an internal volume and includes a first side for the device to contact skin of a patient user and a second side for providing suction; and an array of microneedles protruding from the first side of the body and including a probe encased within a microneedle of the array; and a suction mechanism fluidically coupled to the internal volume of the body at the second side of the body through an opening in the second side. In some embodiments of the device, the skin contact side of the body is opposite to the suction side of the body. In some embodiments of the device, each microneedle in the array of microneedles comprises three or more exterior walls forming a protruding needle structure converging at an apex point, includes a hollow interior defined by an interior wall, and includes an opening to the hollow interior on at least one of the three or more exterior walls of the protruding needle structure, and the hollow interior of each microneedle in the array of microneedles is fluidically coupled to the internal volume of the body through an opening in the skin contact side of the body. Other example embodiments are described that may include additional or alternative features.

Implementations of these and other example embodiments are described herein.

### BRIEF DESCRIPTION OF THE DRAWINGS

FIGS. 1A, 1B and 1C show diagrams and images depicting an example embodiment of an integrated system for a microneedle array sensor patch for continuous measurement of multiple analytes within interstitial fluid, in accordance with the present technology.

FIGS. 1D-1F show diagrams and images depicting another example embodiment of the integrated system for a microneedle array sensor patch for continuous measurement of multiple analytes within interstitial fluid, in accordance with the present technology.

FIG. 1G shows a diagram depicting an example electrode configuration for the example microneedle electrode array of the example embodiment of the microneedle array sensor patch device shown in FIGS. 1A-1C.

FIG. 1H depicts a diagram of the example electrochemical detection modalities that can be employed by the example integrated system shown in FIG. 1A.

FIG. 2 shows a series of user interface produced by a software application of the integrated system operable on a mobile device, in accordance with the present technology.

FIG. 3 shows images and a diagram illustrating an example implementation of multiple analyte detection using an example embodiment of the microneedle array sensor.

FIG. 4 shows a variety of images depicting example microneedle/micro electro pillars fabrication and integration in accordance with the disclosed technology.

FIG. 5 shows a schematic of an example enzymatic multianalyte system and an enzymatic-based sensor system, which feature the immobilization of analytes on example embodiments of the disclosed microneedle arrays.

FIG. 6A shows a diagram of an example embodiment of the microneedle-microelectrodes-microelectronics interface, in accordance with aspects of the present technology.

FIG. 6B shows images of example microneedle electrode array and electronics illustrating an example implementation of the microneedle-microelectrodes-microelectronics interface.

FIG. 7 shows an example embodiment of an in-vivo ISF extraction device, in accordance with the present technology.

FIG. 8 shows an example embodiment of an in-vivo ISF extraction device, in accordance with the present technology.

FIG. 9 shows different views of an example embodiment of an in-vivo ISF extraction device, in accordance with the present technology.

FIG. 10 shows an illustration of example micro-interface components (panel A, left) and images of fabricated microneedles/microelectrodes with Platinum thin films and holes for pogo pin contacts (panel B, right).

FIG. 11 shows a schematic of an example Microneedle (MN) / Micropillar (MP) / Spring-loaded Pin (SP) interface (panel A, left) and images of actual MN/MP/SP connected to wires for in-vitro characterization of the sensing integration (panel B, right).

FIG. 12 shows images of an example microneedle array (panels A and B), images of example fabricated micropillars and micro-masks (panel C), and an image of an example integrated interface including MN-M-pillars and pogo-pins to the E-Board (panel D).

FIGS. 13A-13E show schematics and images depicting an example embodiment of a microneedle with micropillar electrodes connected to electronics in accordance with the present technology, including of example microneedle patches of microneedle arrays.

FIG. 14A shows a diagram of an example embodiment of a single piece microneedle/microfluidic/sensor device for fluid extraction and sensing in accordance with the present technology.

FIGS. 14B and 14C show example results of extraction/glucose sensing experiments using a skin mimicking setup and using a skin liquid setup, respectively.

FIG. 15 shows a diagram depicting an example pillar-based microneedle-type microelectrodes for example tip bio-sensing applications.

FIG. 16 shows images depicting an example implementation demonstrating microneedle skin piercing and fluidic properties.

FIG. 17 shows photographs and SEM images of example microneedles with variety of shapes, sizes and spacing demonstrating the high precision and resolution of the micro-CNC machining technique.

FIG. 18 shows an illustration of an example solid microneedle patch producible using V-Groove and Trace strategy.

FIG. 19A shows diagrams and an image of an example embodiment of a single-piece microneedle/microfluidic channel device with sensor unit integration, in accordance with the present technology.

FIGS. 19B-19D shows images and example data showing an extraction of dyed fluid and in-vitro glucose extraction/sensing.

### DETAILED DESCRIPTION

Currently, commercially available continuous glucose monitoring (CGM) devices are invasive and use relatively long needles (e.g., 5-11 mm) to reach the subcutaneous fat layer under the skin. While the CGM modality has been effective and widely adopted, there are still concerns with use of such needle-type sensor systems for ubiquitous adoption. Thus, a less invasive sensor system would be beneficial for continuous glucose monitoring, as well as continuous monitoring of other analytes, and particularly monitoring of multiple analytes, simultaneously.

Existing wearable microneedle sensor device also suffer from non-optimal performance with respect to the quality of the detectable signal sensed by the microneedle sensor device, as well as for the practicality of a user’s ability to effectively use the microneedle sensor device for the intended duration and at the optimal location. For example, existing skin-wearable microneedle sensor devices lack a robust interface between the microneedle electrodes and electronics that transduce the electrical small-signals corresponding to the electrochemically-detectable analytes.

Disclosed herein is a robust, reusable, and minimally invasive microneedle sensor device including a multi-sensor microneedle electrode array patch interfaced with fully-integrated electronics (and, in some embodiments, a smartphone application) for continuous monitoring of multiplex analytes within the human interstitial fluid (ISF) using an array of microneedles with substantially smaller heights than conventional CGM needle-based sensors. In some embodiments, the microneedle height of the sensor patch ranges from 400-800 µm. The disclosed devices are capable of reuse by providing disposable microneedle array to the reusable electronics, all while ensuring a water-tight connection via a unique electrode-electronics mating scheme.

In some aspects, described below are example embodiments of a completely integrated wearable system for simultaneous monitoring of multiple analytes continuously. Also described are example implementations that provide a first demonstration of a stand-alone integrated wearable system capable of measuring and monitoring multiple analytes within human interstitial fluid (ISF) in a continuous manner. Unlike other existing wearable sensor devices in the market, the disclosed sensor patch is capable of multiplexed sensing of multiple disease biomarker (e.g., diabetes-related analytes or analytes associated with other maladies, including glucose, lactate, alcohol, electrolytes, and other disease biomarkers) within interstitial fluid (ISF), with simultaneously and continuously monitoring.

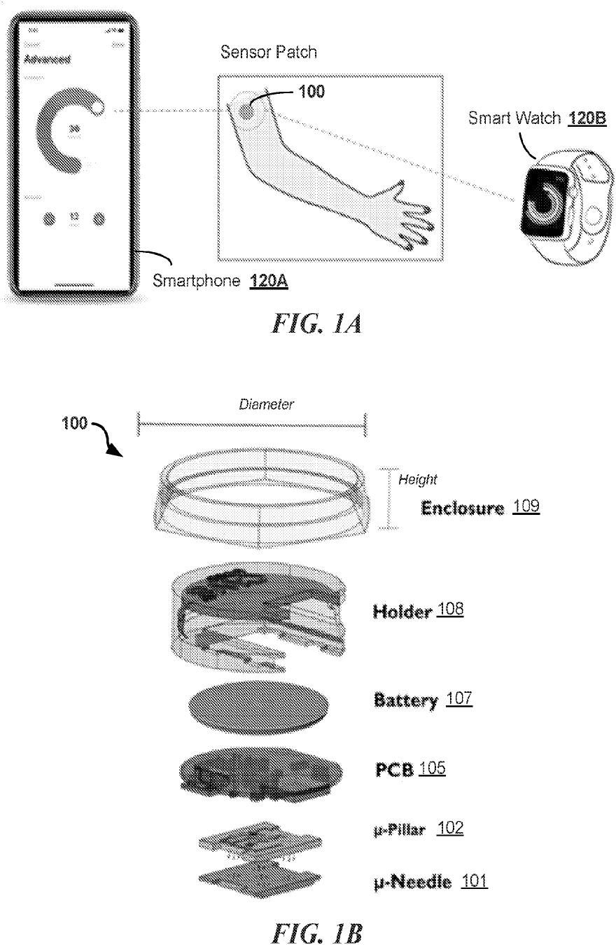

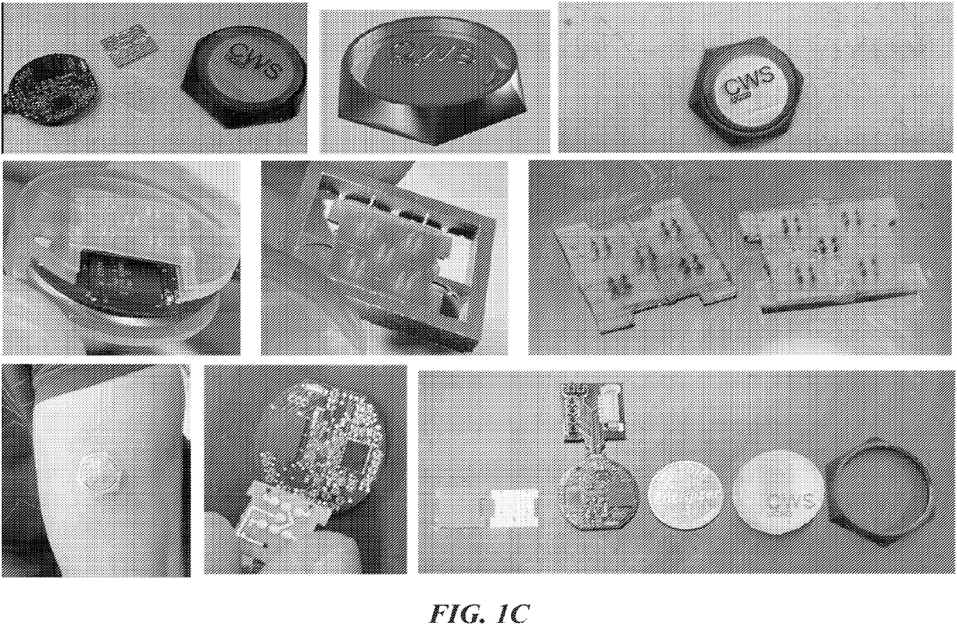

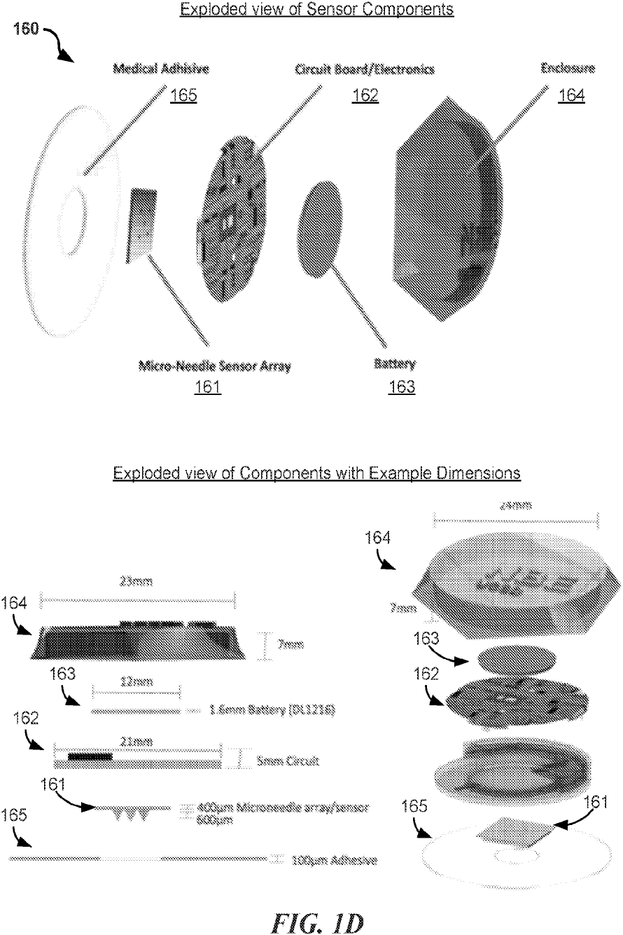

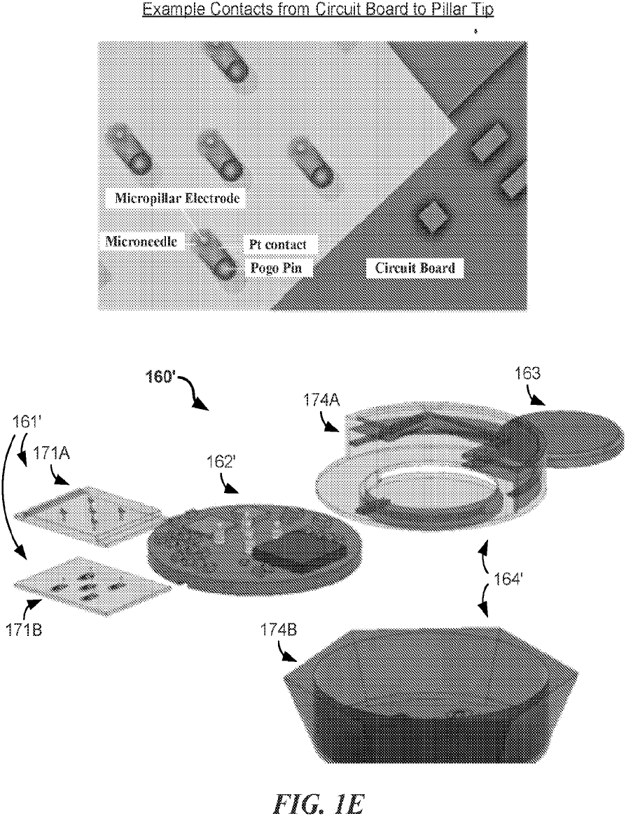

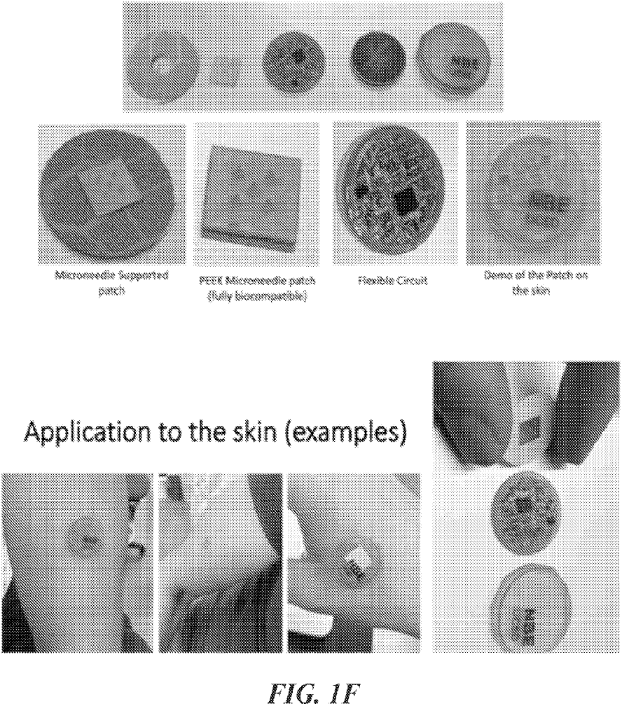

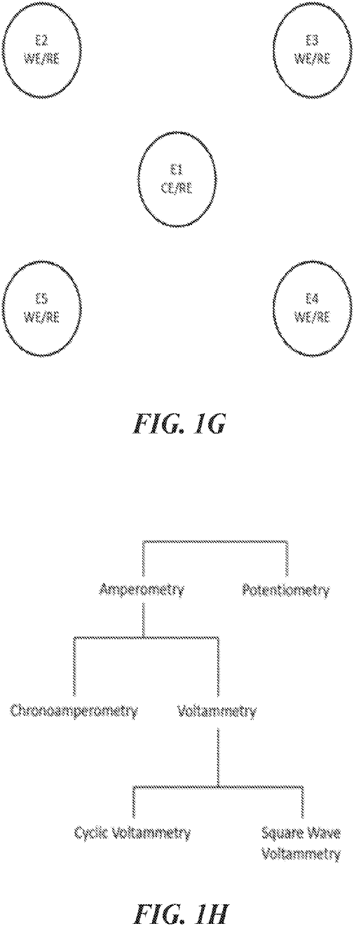

FIGS. 1A-1C show diagrams and images depicting an example embodiment of an integrated system for a microneedle array sensor patch for continuous measurement of multiple analytes within ISF.

FIG. 1A shows a diagram illustrating a microneedle array sensor patch device 100 in wireless communication with a mobile device 120, e.g., smartphone 120A, smartwatch 120B, and/or other device (not shown), such as a tablet, smart glasses, laptop computer, and the like. The mobile device 120 includes a software application (“app”) that is configured to process data provided by the microneedle array sensor patch device 100 and provide a user interface to a patient user of the device 100, e.g., to display data such as present analyte values detected by the microneedle array sensor patch device 100, and/or past analyte values detected by the microneedle array sensor patch device 100. In some embodiments, the microneedle array sensor patch device 100 is in communication with one or more computers via a network of computers (e.g., the cloud), where data can be transferred to the one or more computers through the cloud via the mobile device 120.

FIG. 1B shows an exploded view diagram depicting the components of at least some example embodiments of the microneedle array sensor patch device 100. The example microneedle array sensor patch device 100 includes a microneedle sensor contingent that includes an array of microneedles that protrude outward of the device so as to minimally puncture the skin of a patient user, where an array of micropillar electrodes are disposed in the microneedles to detect target biomarkers in ISF through electrochemical detection and provide an electrical output at an electronic device interfaced with the micropillar array. In some embodiments, the microneedles of the array include a height ranging from 400-800 µm. In some embodiments, the protruding microneedle structure of at least some of the microneedles of the array includes (i) one exterior wall such that the protruding needle structure is of a conical shape, (ii) three exterior walls such that the protruding needle structure is of a triangular pyramid shape, and/or (iv) four exterior walls such that the protruding needle structure is of a rectangular pyramid shape. In some embodiments, the protruding microneedle structure of at least some of the microneedles of the array can include greater than four exterior walls to for other geometries.

As depicted in the diagram of FIG. 1B, the example microneedle array sensor patch device 100 includes multiple component layers that form the device, including a microneedle layer 101 coupled to a micropillar layer 102, which is in electrical connection with an electronic device layer 105 (e.g., a printed circuit board (PCB)) electrically connected to a power source 107 (e.g., battery). The components of the example microneedle array sensor patch device 100 can be contained by a housing structure, which can include a holder 108 and an enclosure 109. The holder 108 can position the microneedles and/or micropillars to protrude outward of the device 100 as well as position at least some of the component layers with respect to each other, and the enclosure 109 can provide an outer structure to secure the device components. Various examples of these device layers and device components are discussed and shown in further detail and/or with variations, below.

FIG. 1C shows multiple images of an example implementation of the example embodiment in FIG. 1B of the microneedle array sensor patch device 100, e.g., demonstrating the sensor patch device 100 in and outside of the housing structure, worn on an example patient user, and separated into its various components like that illustrated in FIG. 1B.

The electronics of the example microneedle array sensor patch device 100 have been designed to measure the small signals that result from microneedle-based sensors. Moreover, the connections between the microneedle sensor contingent and the electronics have been designed to allow connections across the fabrication tolerances of both the microneedles and the electronics, while enabling re-use of the electronics with disposable microneedle arrays, e.g., all while ensuring a water-tight connection. Specifically, the connection between the microneedles and the electronics has been designed so that the end user can easily and reliably attach the disposable microneedle array to the reusable electronics. The unique mating of the microneedles to the electronics also seals the electronics from all outside fluids. The electronics have been designed for low power operation to allow for extended operation from a Li-ion battery, and features either wired or wireless re-charging.

FIGS. 1D-1F show diagrams and images depicting another example embodiment of the integrated system for a microneedle array sensor patch 160 for continuous measurement of multiple analytes within ISF, in accordance with embodiments of the microneedle array sensor patch device 100. The example microneedle array sensor patch 160 provides an epidermal platform for multiplex sensing of disease-related biomarkers, including but not limited to glucose, lactate, alcohol, or other analyte within human interstitial fluid (ISF). Using the example microneedle array sensor patch 160, these example analytes can be measured continuously within the ISF amperometrically transmitted wirelessly via Bluetooth to other wearable and portable smart devices including smart watches and phones. The example microneedle array sensor patch 160 is an integrated system including micro-sensor, electronics components, and packaging components, as depicted in FIGS. 1D-1F.

The disclosed microneedle-based micro-sensors, including the example microneedle array sensor patch 160, are designed to painlessly penetrate through the epidermis accessing ISF found within the dermis. Biocompatible Polyetheretherketone (PEEK) materials can be used to fabricate the microneedles as well as the base of the micropillars. The sensor patch can be developed to be pain-free and reliable for up to 7 days of use on the human body. For example, the secondary electronics component of the example microneedle array sensor patch 160 is reusable and can continuously measure the analyte for up to 7 days without recharging.

Unlike the current state of the art systems, the disclosed example microneedle array sensor system is designed for multiplex sensing of multiple analytes associated with one disease or with multiple related (or unrelated) diseases, providing parameters (such as analyte concentration) of these detected analytes concurrently and continuous over time. Such capability will allow cost effective, real-time monitoring of subjects who may not yet know they have any particular disease (e.g. as the disclosed device can be implemented as a precautionary-monitoring system). Additionally, the example microneedle array sensor patch 160 and its microsensor can be fabricated using a new, unique micromachining method, described herein. For example, the microneedle fabrication method provides tremendous flexibility in fabrication of variety of microneedles with size, shape, spacing, tip geometry, and the patch materials selection (e.g., soft metals such as aluminum and copper, machinable polymers, and some ceramics), e.g., depicted in FIG. 1D. Furthermore, the microsensor fabrication method is highly cost-effective, precisely reproducible (e.g., 1 µm precision), and readily scalable. The fabricated array is applicable to many scenarios including but not limited to on-body electrochemical bio-sensing applications and real time extraction of human body fluids such as Interstitial Fluids (ISF) and blood.

FIG. 1D shows an exploded view (top) of example components of the microneedle array sensor patch 160, and another exploded view (bottom) of example the components with their respective sizes. As shown in this example, the microneedle array sensor patch 160 includes a microneedle sensor array apparatus 161, which is interfaced to the sensor device electronics unit 162, where the sensor device electronics unit 162 is electrically coupled to a power source 163 (e.g., battery). The microneedle sensor array apparatus 161, the sensor device electronics unit 162, and the power supply 163 are enclosed in a water-tight sensor packaging 164 (enclosure), which can couple to an adhesive 165 (e.g., medical adhesive material, like Tegaderm or other). The water-tight sensor packaging or enclosure 164 has an aperture or region providing openings, allows the microneedle sensor array apparatus 161 to protrude outward to interface with a user’s skin.

The bottom diagram in FIG. 1D shows the example components 161-165 of the microneedle array sensor patch 160 for a particular design of the example embodiment with certain size specifications. In the example, the height of the water-tight sensor packaging 164 is configured to 7 mm, and the diameter (or widest length dimension) of the water-tight sensor packaging 164 is configured to 23 mm or to 24 mm. Also, in this example, a battery is used as the power source 163, which includes a height of 1.6 mm and a diameter of 12 mm. The sensor device electronics unit 162 in this example includes a PCB board having a height of 5 mm and a diameter (or widest length dimension) of 21 mm. The microneedle sensor array apparatus 161, in this example, includes a height in a range of 400 µm to 600 µm. The adhesive 165 includes a medical adhesive layer having a height of 100 µm.

FIG. 1E shows a zoomed-view diagram (top) of an example printed circuit and interface contacts for another example embodiment of the microneedle array sensor patch 160 (labeled as microneedle array sensor patch 160') in accordance with embodiments of the microneedle array sensor patch device 100, and FIG. 1E shows an exploded view (bottom) of example components of the microneedle array sensor patch 170. As shown in the bottom diagram for this example, the microneedle array sensor patch 160' includes a microneedle sensor array apparatus 161', which comprises a microneedle array layer 171A and a micropillar electrode layer 171B that interfaces with the microneedle array layer 171 such that an array of micropillar electrodes of the micropillar electrode layer 171B fit within hollow cavities of respective microneedles of the array. The microneedle sensor array apparatus 161' is interfaced to the sensor device electronics unit 162', where the sensor device electronics unit 162' is electrically coupled to a power source 163 (e.g., battery). The microneedle sensor array apparatus 161', the sensor device electronics unit 162', and the power supply 163 are enclosed in a water-tight sensor packaging 164' (enclosure), which can couple to an adhesive component like the adhesive 165. In this example, the water-tight sensor packaging or enclosure 164' can include a holder or housing structure 174A that can secure at least some of the components of the microneedle array sensor patch 160' (e.g., including the power source 163, the sensor device electronics unit 162', and/or at least some of the components of the microneedle sensor array apparatus 161'). The water-tight sensor packaging or enclosure 164' can include an enclosure to provide a structure that secures the holder or housing structure 174A within, while allowing the microneedle sensor array apparatus 161' to protrude outward to interface with a user’s skin.

FIG. 1F images of example sensing components for the microneedle array sensor patch 160 and of an example implementation applied to a user’s skin. The panels of images of FIG. 1F show an example implementation of the microneedle array sensor patch 160 (in assembled and unassembled views) used in a demonstration for multi-analyte continuous monitoring.

In various embodiments like those shown in FIGS. 1D-1F, the microsensors micropillars can fabricated using a scalable micro-computer numerical control (CNC) method in accordance with the present technology. Pillars with desired thickness, diameter, and height are fabricated preciously/reproducibly (e.g., 1 µm precision and reproducibility) with at least one working electrode, and one counter/reference electrodes. In some embodiments of the disclosed CNC method, the working electrode is functionalized using an electropolymerization of o-Phenylenediamine (OPD)/Glucose oxidase (GOx) mixture physically immobilizing the GOx within the PPD layer. The PPD layer is then covered with a precisely controlled thick films of Polyvinyl chloride (PVC) and polyurethane (PU) which act as antibiofouling layers.

The constructed wearable electronic system is reconfigurable which facilitates the measurement of multiple analytes and supports multiple sensor configurations. A quick connect interface has been designed to quickly replace the microneedle sensing array after its useful lifetime. The wearable sensing system can be configured to operate as a two- or three-electrode potentiostat to provide the best results. One of the example advantageous features of the electronics for the example wearable electronic sensor system is the capability to measure multiple analytes by sequencing different combinations of electrodes in the microneedle array. The system is capable of both local data analysis and real time streaming of data to the companion mobile application. The mobile application allows the system to be reconfigured wirelessly to adapt to changing conditions or new microneedle arrays.

As shown in FIGS. 1D-1F, the overall design and integration of the electronics with the microneedle/microsensor components for the on-body sensing application is also another novel aspect of this work. In some embodiments, for example, the sensor is composed of sensing components made by microneedle/microelectrodes, which are designed to penetrate the skin accessing ISF safely and painlessly. The microsensor component is electronically connected to a printed circuit assembly through a quick connect interface. A microcontroller on the printed circuit assembly runs firmware that functions as both a potentiostat and wireless radio. This allows the sensed data to be sent to a smartphone running with application (“app”) software.

In some implementations, for example, the example microneedle array sensor patch 160 and 160' can be attached to the skin with microsensors array penetrating through the skin and measuring the analytes of interest within the ISF electrochemically. An electrochemical measurement can be performed every 5 minutes (or less, i.e., more frequent) over the sensor lifetime, e.g., which can be 7 days. After the sensor lifetime (e.g., 7 days in some implementations), the disposable microneedle array can be replaced. The example results of each measurement are transmitted through Bluetooth to the companion smartphone/smartwatch and are compiled into a graph. This graph tracks the fluctuations in the concentrations of the various diabetically relevant analytes over time giving the sensor wearer and insight to the trend of their analyte concentration within their body over time. Such insight can spark actionable adjustments to the wearer or/and other potential integrated systems, such as insulin pump, etc.

The sensing component has been developed for reproducibility and stability of the sensor towards 7 days of constant functionality. The electronics have been designed, and design files have been prepared for the fabrication of prototypes of the sensor device. The mobile application is communicating with stand in Bluetooth devices.

The example microneedle array sensor patch 160 and 160' can be used for various applications. One example primary use of the epidermal sensor patch is in continuous monitoring of glucose levels within ISF without causing irritation or pain for the user. Furthermore, the example sensor is designed for sequential monitoring of a first analyte (e.g., glucose) with additional analytes (e.g., alcohol, lactate, and/or other analytes) in using the same disposable patch. The example microneedle array sensor patch 160 and 160' can provide a platform for sensing almost any biomolecule of interest compatible with enzymatic electrochemical sensing. This relates to the capability of electronics for nanoampere sensitivity, low noise, and low power operation as well as the sensor which provides wide linear detection ranges, high sensitivity, selectivity and stability through the immobilization protocols applied.

### Electronics Firmware and Remote Software Application

In some embodiments, the electronics of the various embodiments of the microneedle array sensor patch device 100 includes firmware, which runs on an embedded microcontroller, that is operable to control the detection measurements conducted on the example embodiments of the microneedle array sensor patch device 100, as well as perform data analysis, transmit data, and manage system power. In such example embodiments, the microcontroller can include an embedded Bluetooth Low Energy (BLE) radio to transfer data between the sensing device and a mobile software application on a connected device, e.g., the app on the mobile device 120. The mobile software application can provide a flexible measurement control interface, real time data visualization, data analysis, and data export features.

In implementations, for example, for the sensor patch to be capable for detection of multiple analytes continuously, as a system, every major component of the system is required to work in harmony with each other. The electronics hardware (e.g., PCB), the electronics firmware, and the mobile software application (e.g., on the smartphone 120A and/or smartwatch 120B) showing the sensing results are all designed and developed to address that requirement. Similarly, the sensing components are a match for sensing multiple analytes within the ISF, e.g., including but not limited to the following analytes: glucose, lactate, alcohol, and electrolytes.

### Electrode Configurations

FIG. 1G shows a diagram depicting an example electrode configuration for an example microneedle electrode array of any of the example embodiments of the microneedle array sensor patch device 100. In the example shown in FIG. 1G, there are four electrodes that are operable as a working electrode (WE) and/or reference electrode (RE), which are arranged about an electrode operable as a counter electrode (CE) and/or reference electrode (RE).

While other electrode configurations are possible, including more (or less) electrodes, in the example embodiment shown in FIG. 1G, the system’s electrodes can be reconfigured for amperometric electrochemical measurements in the following arrangements.

### Example A: Four Independent ‘2 Electrode’ Amperometric Measurements

In this example, four analytes can be measured in sequence. E1 is a common counter electrode, E2-E5 are independent working electrodes. In this configuration, four different analytes can be measured in sequence.

### Example B: Three Independent ‘2-Electrode’ Amperometric Measurements

In this example, three analytes can be measured in sequence. E1 is a common counter electrode, any two (E2-E5) electrodes can be selected to have their signals combined. This signal augmentation allows smaller concentrations of analytes to be detected. In this configuration the remaining 2 working electrodes can be used to measure other analytes.

### Example C: Two Independent ‘2-Electrode’ Amperometry Measurements

In this example, two analytes can be measured in sequence. E1 is a common counter electrode, any two sets of two electrodes (pick from E2-E5) can be selected to have their signals combined. This signal augmentation allows smaller concentrations of analytes to be detected. This mode would be used if two small concentration analytes need to be measured.

### Example D: Two Independent ‘2-Electrode’ Amperometric Measurements

In this example, two analytes can be measured in sequence. E1 is a common counter electrode, 3 electrodes are chosen (pick from E2-E5) to have their signals combined. This signal augmentation allows smaller concentrations of analytes to be detected. The remaining electrode is used to measure a different analyte. This mode would be used if one low concentration analyte needs to be measured and one high concentration analyte needs to be measured.

### Example E: One ‘2-Electrode’ Amperometric Measurements

In this example, one analyte can be measured. E1 is a common counter electrode, the signals from the remaining four electrodes (E2-E5) are combined. This signal augmentation allows extremely small concentrations of analytes to be detected. This mode would be used if one very low concentration analyte needs to be measured.

### Example F: One ‘3-Electrode’ Amperometric Measurements; and/or Two ‘2-Electrode’ Amperometry Measurements. 3 Analytes Measured in Sequence

In both the ‘3-electrode’ and ‘2-electrode’ configurations, E1 is used a common counter electrode. For the One ‘3-electrode’ configuration, any remaining electrode can be selected to be the working electrode and reference electrode. The remaining two electrodes are used as independent working electrodes in a ‘2-electrode’ configuration.

For the Two ‘3-electrode’ amperometric measurements. 1 ‘2-electrode’ amperometry measurements. 3 analytes measured in sequence.

In both the ‘3-electrode’ and ‘2-electrode’ configurations, E1 is used as a common counter electrode. A single electrode is selected to be a common reference for both ‘3-electrode’ configurations, any remaining electrode can be selected to be the working electrodes in the 2 ‘3-electrode’ configurations. The remaining electrode is used as independent working electrodes in a ‘2-electrode’ configuration.

### Example G: Three ‘3-Electrode’ Amperometric Measurements

In this example, three analytes can be measured in sequence. E1 is a common counter electrode. A single electrode is selected to be a common reference for both ‘3-electrode’ configurations, any remaining electrode can be selected to be the working electrodes in the 3 ‘3-electrode’ configurations.

### Multi-Modal Measurement Configuration

In various implementations, for example, the system combines different electrochemical detection modalities, including potentiometry, cyclic voltammetry (CV), fast scan cyclic voltammetry (FSCV), square wave voltammetry (SWV), chronoamperometry, and more towards the detection of different target analytes (e.g., metabolites, electrolytes, hormones, etc.).

FIG. 1H depicts a diagram of the example electrochemical detection modalities that can be employed by the integrated system, e.g., shown in FIG. 1A, including the various embodiments of the microneedle array sensor patch device 100.

A potentiometric measurement technique is one where the open circuit potential of the electrochemical cell is directly measured. This potential is measured between the reference electrode and the working electrode. This is contrasted with the amperometric family of measurements that measure current while controlling the cell potential, for example.

Chronoamperometry is a powerful tool for measuring diffusion-controlled reactions. In chronoamperometry the potential is stepped at the beginning of the experiment and then remains constant throughout the duration of the measurement. The current that results from this stimulus is plotted as a function of time.

Voltammetry techniques vary the potential as a function of time. The resulting current is plotted as a function of potential. For example, CV sweeps the potential of the cell linearly across a voltage range, while FSCV does this at a faster rate. SWV uses a square wave superimposed over a staircase function to provide a sweeping measurement that provides two sampling instances per potential. As a result of this sampling technique, the contribution to the total current that results from non-faradic currents is minimized. Like CV, the current is plotted as a function of potential.

### Example Measurement Parameters

Each measurement technique has a different set of required parameters. In the example implementations, for the various described measurement techniques, the system can be constrained by the following parameters.

The system is capable of biasing electrochemical cells between -1.2 V and +1.2 V. The system is capable of detecting sub-nanoamp currents using an ADC. The maximum ADC sample rate is 800 kHz. The total measurement duration is configurable and can range from 1 second to 24 hours. Yet, other voltage ranges, sampling rates, and measurement durations are possible.

In some example configurations, 60-second measurements are used and are repeated every 5 minutes. In this 5-minute span, four independent measurements can be taken. If each measurement in this configuration is measuring a different analyte, each analyte will be measured 288 times over a 24-hour period.

The electronics of the microneedle array sensor patch device 100 can be configured to communicate with a remote device, such as the example mobile device 120, e.g., using Bluetooth Low Energy (BLE), such that an ‘app’ resident or operable on the mobile device 120 can receive raw data, pre-processed data, and/or processed data associated with the detected signals by the electronics of the microneedle array sensor patch device 100. For examples using BLE, BLE provides a power efficient method of sending data between the electronics and the app. In an example implementation, the app was implemented in Swift and runs on a modern smartphone (e.g., iPhone 7 - current). The app can be used for all system configuration. This includes the electrode assignment, measurement configuration, and measurement parameters detailed above. In addition to this configuration the app is used to display and record measurement data. The app also reports the remaining battery life of the system. The app is used to convert the voltages and currents measured by the electronics into the concentrations of the various analytes being measured.

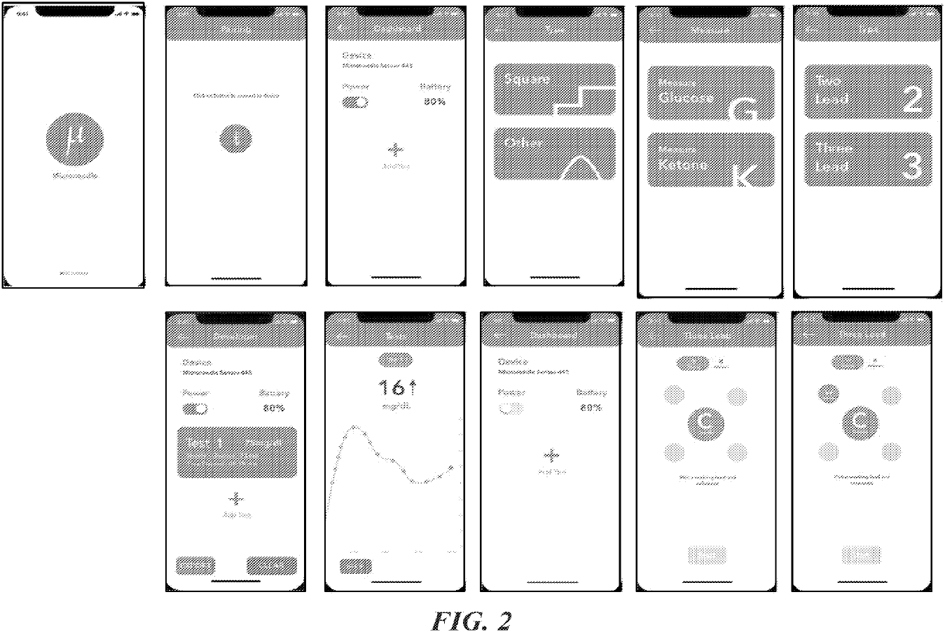

FIG. 2 shows a series of example user interface produced by a software application of the integrated system operable on a mobile device, in accordance with the present technology. The diagrams of FIG. 2 illustrate the app’s iOS app pages, first iteration, showing Bluetooth connectivity, device battery life, experiment configurations, and resulting display.

### Example Implementations for Multiple-Analyte Detection and Simultaneous Monitoring

For example, for the individual sensors to work in the same environment measuring different analytes, working electrodes must be highly selective avoiding any interference with other analytes as well as the sensor being cross-talk-free towards each other and their perspective sensing byproducts. Hence, the disclosed microneedle electrode sensor array patch device 100 can be configured to provide a unique combination of mediator free and mediated biocatalytic sensing electrode where mediators such as Prussian blue are used towards minimizing the sensing potential closer to the open circuit potential of the system which ultimately avoids potential interferences. Furthermore, sequential sensing with short 1-5 minutes intervals is programmed to the PBC to minimize any potential cross-talking among sensors.

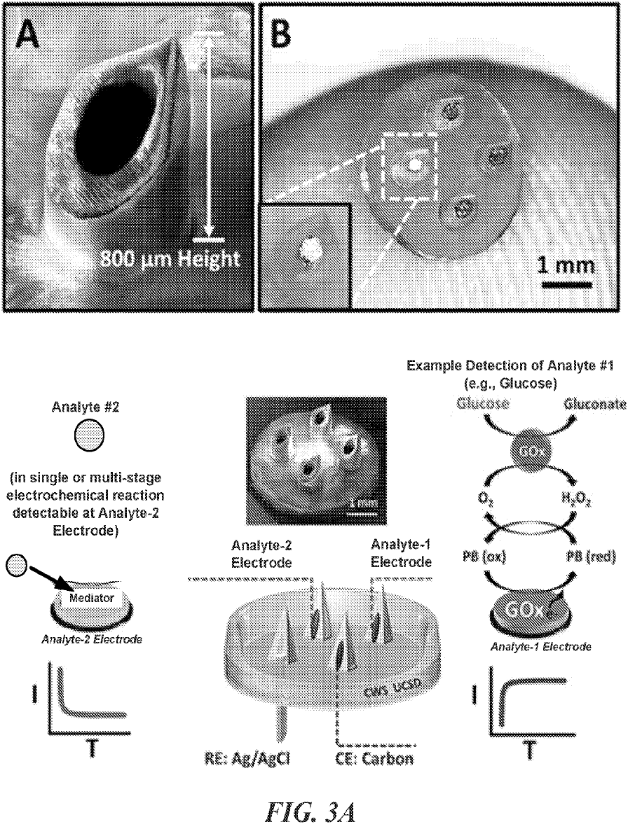

One example of simultaneous monitoring is shown in FIG. 3, reporting monitoring of a first analyte (e.g., glucose) and a second analyte, which can also include a third analyte and a fourth analyte, using an example embodiment of the microneedles, e.g., implemented in an in-vitro setup. As shown in this example data, no significant cross-talking among the sensor needles was observed for multiple scenarios, in which glucose and the other analytes were present in the environment. This strongly supports workability of such complex sensing platform towards on-body applications.

FIG. 3 shows images (above) and a diagram (below) illustrating an example implementation of multiple analyte detection using an example embodiment of the microneedle array sensor, which is monitoring at least two analytes (e.g., analyte #1 and analyte #2), at two working electrodes (e.g., analyte-1 electrode and analyte-2 electrode, respectively). In the example, both analyte-1 electrode and analyte-2 electrode are modified (e.g., by a reactive mediator, catalyst, etc.), although the microneedle array sensor need not modify the electrodes based on conditions of the detecting electrodes and/or the desired analyte to be detected. Also, in the diagram of FIG. 3, the analyte #1 is illustrated for glucose, as an example, showing the simultaneous detection of glucose and analyte #2 on the same sensor array.

### CNC-Based Fabrication Methods

Also disclosed are CNC-based fabrication techniques for producing microneedle sensor arrays. In some embodiments, the microneedle patch and microelectrodes are fabricated by using a micro-Computer Numerical Control (CNC) micromachining method in accordance with the disclosed technology. This microneedle fabrication method provides a tremendous flexibility in fabrication of variety of microneedles with size (e.g., diameter from 200 µm with holes diameter from 50 µm, and height from 200 µm), shape, spacing, tip geometry, and materials selection (e.g., soft metals such as aluminum, 316L stainless steel and copper, machinable polymers, and some ceramics). Furthermore, the disclosed CNC-based fabrication method is highly cost-effective, precisely reproducible (e.g., 1 µm precision), and readily scalable.

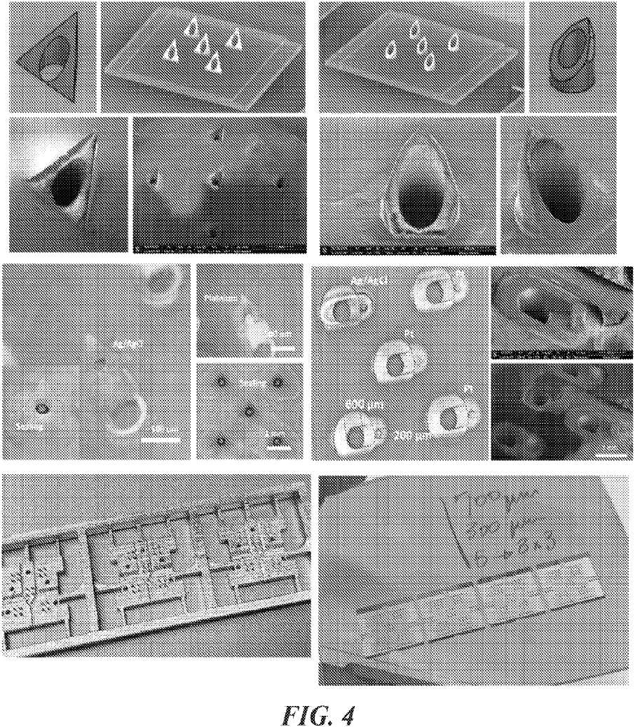

FIG. 4 shows a variety of images depicting example microneedle/micro electro pillars fabrication and integration. As shown in the FIG. 4, high precision micropillar electrodes provide exact sensing surface area among sensing electrode without which sensing would not be reproducible. The example micropillar electrodes (also referred herein simply as “micropillars”) are can be produced by high precision stereolithography printing or micro CNC technique. To make each micropillar conductive, for example, chromium, platinum, and silver are sputtered on both sides of the pillar patches which are either masked or post treated for electrical isolation of each electrode to become individually addressable. Furthermore, the silver layer is etched away from the working electrodes by drop casting HNO₃ 6 M for 1 minute and the silver on the reference electrode is turned into Ag/AgCl by drop casting of FeCl₃, 0.1 M for another minute. Signal amplification for each working electrode is implemented by fabricating an increased number of microneedle/micropillars (e.g., ranging from 10-20) for each individually addressable working electrode.

### Array Fabrication Involving Enzymatic-/Bioaffinity-Based Electrodes

The disclosed sensor array design allows combination of different types of target analytes based on different detection modalities, using micropillar electrodes functionalized with different types of receptors. Sensitivity, selectivity, detection range, and stability of each sensor is carefully optimized using engineered immobilization protocols and combination of materials used. Each microelectrode can be coated with a unique combination of metal substrate (e.g., Au, Pt), carbon based composites (e.g., graphene, graphite) oxidase and dehydrogenase enzyme (e.g., glucose oxidases, 3-hydroxybutyrate dehydrogenase etc.) cross linker, polymeric coatings (e.g., PVC, Chitosan, Polyurethane, etc.) in a way that it fulfills requirements of multiplex continuous sensing of the low concentration aforementioned analytes within the human ISF.

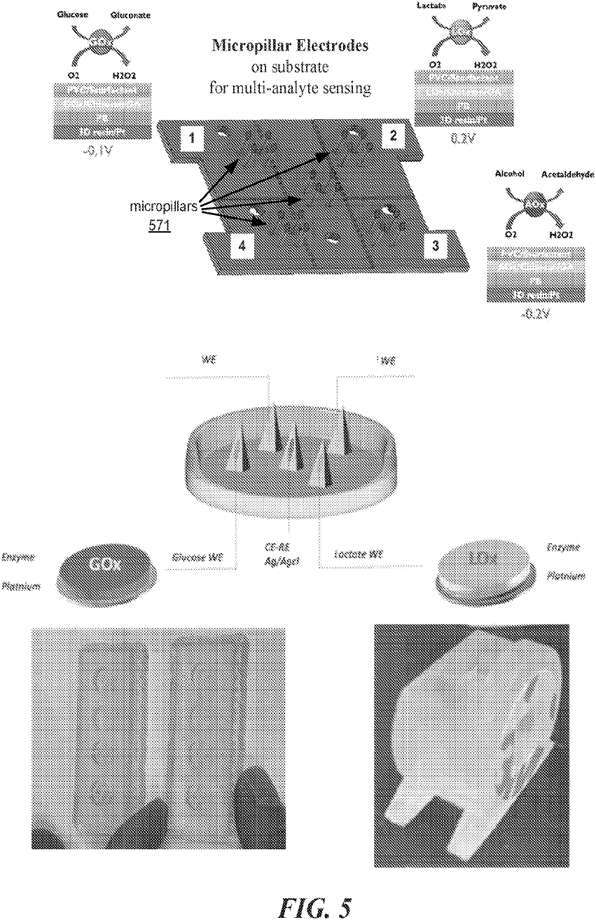

FIG. 5 shows a schematic of an example enzymatic multi-analyte system sensor system, which feature the immobilization of analytes on example embodiments of the disclosed microneedle arrays. In the example, there are four sensing micro-electrodes (e.g., individual arrays of micropillars 571 configured for sensing different analytes 1, 2, 3 and 4) and one reference electrode to measure aforementioned analytes within ISF simultaneously, e.g., with a five minutes interval between each sensing. Each electrode can include a platinum or gold enzymatic sensor with proper stabilizing polymeric coatings which allows continuous detection of analytes continuously for up to 7 days. Antibiofouling properties of the microsensors is of utmost importance when it comes to the continuous monitoring. Therefore, specific antibiofouling coatings of a unique combination of polymers such as Polyvinyl chloride (PVC), p-Phenylenediamine (PPD), Polyurethane (PU) and chitosan or zwitterionic polymers with excellent antibiofouling properties, and cross linkers such as Polyethylene glycidyl di-ether (PEGDE) and Glutaraldehyde (GA) is developed towards keeping the enzymes in a stable environment while keeping the sensitivity and detection range of the sensors within ideal. A unique sequence of immobilization techniques such as dip casting, electrochemical deposition and drop casting using uniquely fabricated immobilization cells are used to implement uniformly reproducible immobilization protocols for each sensor in a scalable fashion.

### Microneedle/Microelectrodes/Microelectronics Interface

The disclosed integrated system for microneedle array sensing platform also includes an effective interface of the electronic interface and the microneedle sensor array using spring-like pogo-pin connectors. One of the major challenges of microneedle based wearable sensors is related to the encapsulation and insolation of the sensor electrodes in a way that there is not interference of the sweat as well as cosmetic skin products during the sensor performance. Yet, at the same time, there must be a way to electrically connect these sensor electrodes to a re-usable electronic module. Although it is possible to encapsulate an entire microneedle array + electronics system to prevent moisture intrusion, the electronics are relatively expensive, thus motivating the need for re-use of the electronics.

Unlike any other microneedle sensor systems, the disclosed integrated microneedle sensor patch-electronics device provides a unique connection between the microneedle/micropillar sensor array and a re-usable electronic module, which addresses the aforementioned challenges. Such connection is based on a unique spring-like pogo-pin connectors. In addition to the physical insolation of the micropillars with hollow microneedles, such connection must have a low impedance to pass the signals between the electronics and MN array without any distortion. The connection must also allow end users to easily and reliably align and connect the MN array to the electronic connection points. The connection mechanism must also seal the electronics (e.g., IP64) to prevent sweat, rain, or other fluids from damaging the electronics.

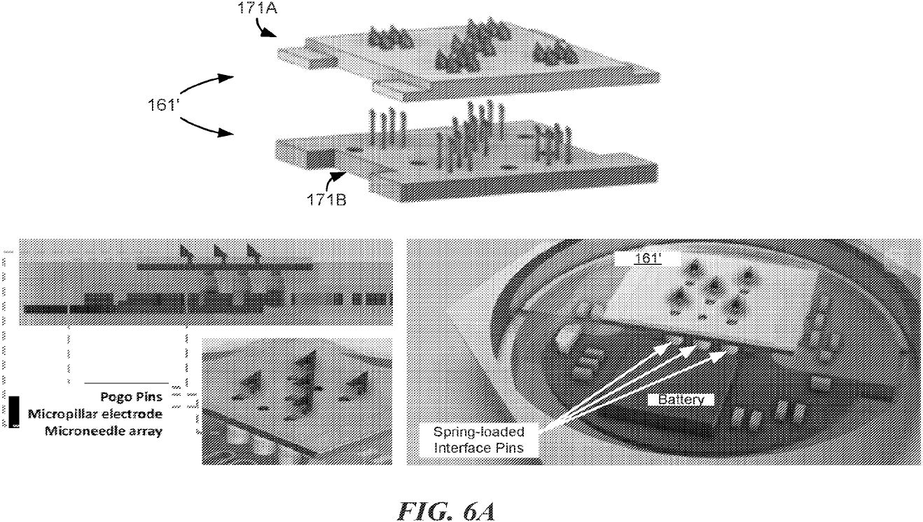

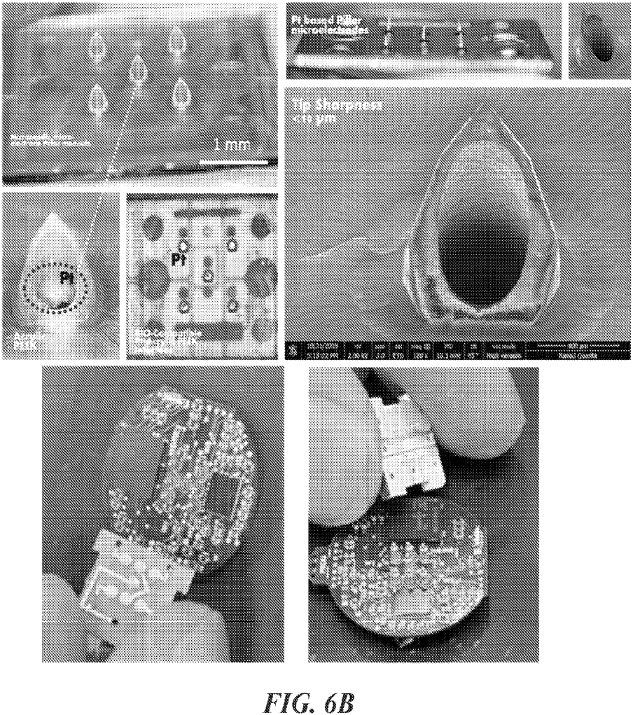

FIG. 6A shows a diagram of an example embodiment of the microneedle-microelectrodes-microelectronics interface for embodiments of the microneedle array sensor patch device 100. In the example embodiment of the microneedle-microelectrodes-microelectronics interface, the integrated interface including three primary components comprising a hollow microneedle array, a complimentary microelectrode micropillar array, and a pogo pin based electronic contact provide a unique design for an electrode-electronics interface allows for important requirements and advantages, including but not limited to improved signal-to-noise ratio, extended power source life and energy conservation, reduced electrical circuit failure, opportunity for conformal coverage, etc.

In the example embodiment, a two-sided printed circuit board (PCB) is used to integrate the electronic components. On the bottom of this PCB, a plurality of surface-mount, spring loaded pins are mounted, e.g., seven pins. These pins are used to make electrical connections between the microneedle sensing array and the electronics. Of the example seven pins, five are used for sensing and two are used to create an automatic power switch. The two pins used in the power switch provide a conduction path through the microneedle array. When this conduction path is completed with the insertion of a microneedle array, the device turns on. When the microneedle array is removed this connection is broken and the device turns off. This automatic power switch is used to increase battery life and protects against over discharging the Li-ion battery.

FIG. 6B shows images of the example integrated microneedle electrode array and electronics of FIG. 6A, illustrating an example implementation of the microneedle-microelectrodes-microelectronics interface.

### Individually Addressable Micropillar Microelectrodes for Multi-Analyte Detection

In various embodiments, the disclosed microneedle array sensor platform is configured to ensure that each microelectrode sensor is electrically isolated and reliably connected to the electronics pogo-pins. As discussed above and throughout this patent disclosure, the connection from the sensor tip to the electronics is critical to be stable and robust for the sensor patch to capture low-noise and accurate signals. The disclosed technology can achieve this, in some embodiments, by creating channels on the backside of the micropillar electrode base and create holes which connects both sides of the base electronically after sputtering metals on both sides of the microelectron micropillar component. The micropillars are then protected by fabricated microelectrodes that match them accurately in terms of the micropillars position on the base as well as the exact holes. Micropillar microelectrodes then are sealed using innovative methods such as use of micro-heat shrink sealants, and photocurable/biocompatible resins which are drop casted to the base of the micropillars and sucked into the gap between the micropillars and microneedle holes by capillary forces followed by UV/heat treatment curing.

### In- Vivo ISF Extraction Device Embodiments of the Microneedle Array Sensor Patch

In some aspects of the present microneedle array technology, example embodiments of an in vivo ISF extraction microneedle array device and high-precision micro-fabrication method are disclosed. Notably, the example embodiments for the in vivo ISF extraction devices can also be fabricated using other methods (e.g., 3D printing).

In some embodiments in accordance with the present technology, an ISF extraction device includes an array of hollow microneedles, a chamber adjacent to the array, and an assembly to facilitate ISF extraction into the chamber through a suction mechanism. In some examples, the device body is cylindrical, e.g., coin-like, with the microneedle array in the central region of the device. The device is applied with the microneedle array side on the subject’s skin. The skin-contact side of the device may be flat from the base of the microneedles to the circumferential edge thus allowing as much of the entire height or length of the needles to penetrate the skin. Alternatively, for example, the skin-contact side can be fabricated to have a ring around the circumferential edge that rises to a fraction of the height of the needles, which ring could ease compression of the skin and collection of ISF within the circle.

In some embodiments, the suction mechanism can be via syringe-like or bellows-like spring action, or an external pump. The extracted ISF is collected in the chamber. The side opposite the skin-contact side is solid in a bellows-like spring embodiment, and has a tube opening in the external pump embodiment.

In various embodiments, the ISF extraction microneedle array device can employ the sensor probe within the microneedle array to sense one or more analytes in the extracted ISF. For example, in some embodiments, the array of microneedles includes a microneedle array sensor unit, which includes an array of electrode probe structures, each disposed within the hollow interior of a respective protruding needle structure, wherein an electrode probe structure of the array is configured to interact with an analyte that comes in contact with the electrode probe structure via the opening to the hollow interior to produce an electrical signal indicative of an electrochemical reaction involving the analyte. In some examples, the array of electrode probe structures includes a first electrode probe structure that is configured to interact with a first analyte that comes in contact with the first electrode probe structure via the opening to the hollow interior to produce a first electrical signal indicative of an electrochemical reaction involving the first analyte, and a second electrode probe structure that is configured to interact with a second analyte that comes in contact with the second electrode probe structure via the opening to the hollow interior to produce a second electrical signal indicative of an electrochemical reaction involving the second analyte. In various embodiments, the device includes or can interface with an electronics unit to be in electrical communication with the array of electrode probe structures, the electronics unit comprising a power source, a data processing unit, and a wireless transmitter.

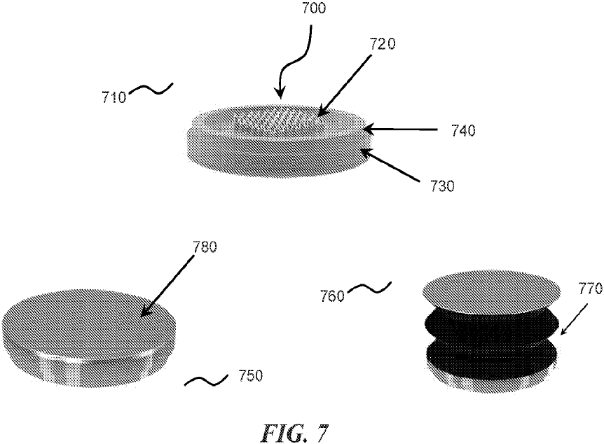

Example in vivo ISF extraction microneedle array devices are illustrated in the FIGS. 7-9. These example devices have been fabricated where the microneedles in the array are, for example, 800 µm tall with and without rings (as tall as half the microneedle height). In one embodiment, the array comprises 25 microneedles total with “every other” spacing between the needles to avoid the “bed of needles” effect. For the external pump embodiment, one could use a hand bulb instead or an electronic vacuum pump. The chamber can connect to the pump via a tube of standard diameter (e.g., plastic tubing from McMaster along with a push-to-connect adapter, with ¼ʺ OD). O-rings are used to seal the device assembly. The device could include filter paper inside the chamber to assist with passive diffusion.

FIG. 7 shows a view 710 of a skin contact side of an embodiment of an in-vivo ISF extraction device 700 according to the technology disclosed herein. As shown in view 710, device 700 includes an array of hollow microneedles 720 on the skin contact side of the device, a chamber 730 adjacent to the array and configured to receive ISF from the array in response to the ISF extraction from the body of a person by extraction means through a suction mechanism. As shown in FIG. 7, device 700 includes a ring 740 around the circumferential edge of the chamber 730 that rises to a fraction of the height of the microneedles. Ring 740 can ease compression of the skin and facilitate collection of the ISF. Views 750 and 760 in FIG. 7 show a suction side of the device 700. In this example, the suction mechanism of the device 700 employs syringe-like or bellows-like spring action. View 750 corresponds to the configuration of the device 700 wherein the bellow 770 is compressed and pressed against the chamber 730 by the base 780. View 760 corresponds to the configuration of the device 700 wherein the bellow 770 is expanded leading to suction of the IFS through the microneedles 720 into the chamber 730 due to the expanded volume of the bellow 770. Expansion of the bellow 770 can be facilitated by a spring housed inside the bellow.

FIG. 8 shows a view 810 of a skin contact side of an embodiment of an in-vivo ISF extraction device 800 according to the technology disclosed herein. FIG. 8 also shows a view 820 of a suction side of the device 800. Device 800 employs a suction mechanism based on using an external pump or a medical inflation bulb to transfer ISF from the body of a person into the chamber 730 of the device 800 through the array of microneedles 720. As shown in view 820, device 800 incorporates a port 830 adapted for connection to a vacuum line (e.g., a tube) of a pump and/or connection to a medical inflation bulb. Removal of air from the port 830 area by the pump or due to expansion of the inflation bulb leads to suction of the ISF through the microneedles 720 from the body of a person into the chamber 730.

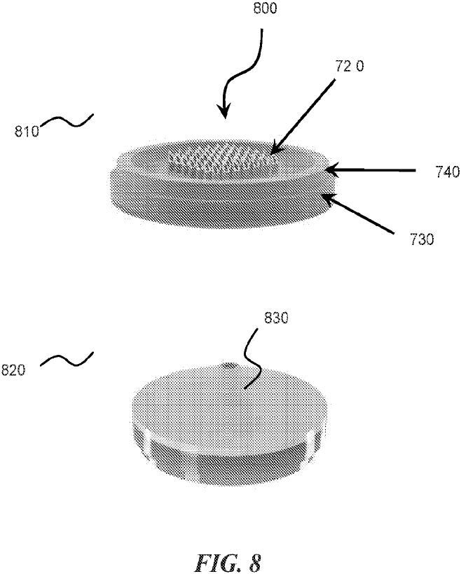

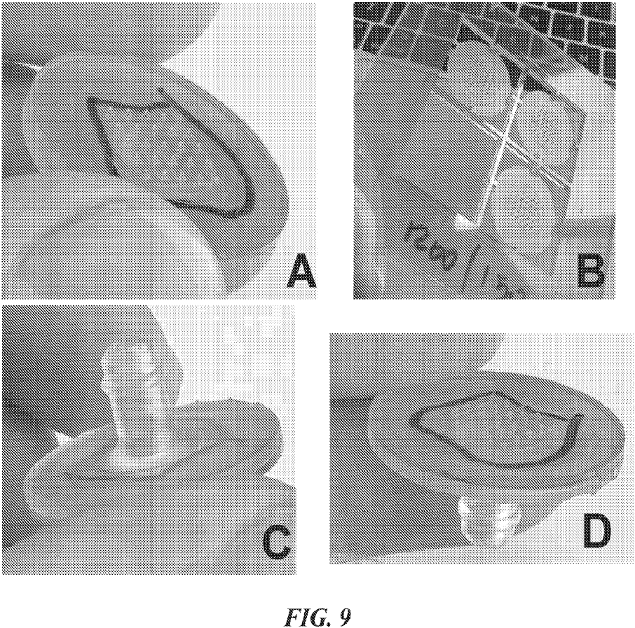

FIG. 9 shows several views of an implementation of the device 800. Panel A in FIG. 9 shows a view of the skin contact side of the device 800. Panel B in FIG. 9 shows a view of the skin contact side of several devices 800. Panel C in FIG. 9 shows a view of the suction side of the device 800. Panel D in FIG. 9 shows a perspective view demonstrating both the skin contact side and the suction side of the device 800.

Further example embodiments, implementations and discussion of the disclosed microneedle sensor platforms are provided below.

For example, the disclosed systems, devices and methods for a micro-sensing interface can be implemented for wearable sensing and therapeutic applications. The interface is composed of microneedle arrays which puncture the skin, micropillar microelectrodes for sensing applications, and electronics with pogo pin contacts to the micro-electrode platform. The interface allows the single use micro electro platforms to be reliably aligned and connected to the reusable electronics. The electronics are capable of testing the connections between the multi-channel and the electronics in addition to their primary function of monitoring the concentrations of various biomedically relevant analytes. The disclosed interface includes a small-size, robust and repeatable connection mechanism, and waterproof connections.

The connection between the microelectrode sensors and the electronics is an important for the operation of the sensing system. Without this connection, the system may not be able to perform its primary function: sensing. The disclosed interface has been designed to allow a layperson to make this connection quickly and reliably. For example, the complete connection can be made in less than five seconds and the electronics provide a visible indication of a successful connection through an integrated self-test feature. The electrical connection between the microelectrode platform and electronics is made with miniature spring-loaded contacts. These contacts are permanently mounted to the custom printed circuit assembly. The alignment between the microelectrode platform and electronics is enforced through a custom plastic housing.

The example micro-interface is a platform for wearable applications where penetration through the skin is necessary towards providing micro electrodes access to the sub-epidermal fluids such as Interstitial Fluid (ISF), and blood. Such access is done in a minimally invasive manner with negligible pain induction to the wearer while being reliably leakage proof and electronically robust.

Some example embodiments of the interface are composed of three components: a microneedle array, micropillar microelectrodes, and the electronics, as depicted in FIG. 10 (panel A). The micropillar microelectrode platform is fabricated by micromachining followed by masking and sputtering of chromium, platinum or gold, and silver at a controlled timing and sputtering pressure, e.g., depicted in FIG. 10 (panel B). For example, the silver microelectrodes are then etched to platinum or gold and silver chloride as working and reference electrodes, respectively. Furthermore, the microelectrodes are functionalized for continuous monitoring of variety of biomarkers through enzymatic-based and affinity-based detection.

FIG. 10 shows an illustrative diagram of example micro-interface components (panel A, left) and images of fabricated microneedles/microelectrodes with Platinum thin films and holes for pogo pin contacts (panel B, right). In the diagram shown in panel A (left) of FIG. 10, the example microneedle-microelectrodes-microelectronics interface includes a hollow microneedle array, a microelectrode micropillar array, and an array of surface-mount, spring-loaded electronic contact structures that electrically interface the micropillar electrodes directly to the particular contact points on the electronic circuit board (e.g., PCB board) in a manner that improves signal-to-noise ratio of the detected signal by the respective micropillar electrode, can extends power source life and conserve energy of the device, reduce electrical circuit failure, etc. In some embodiments, for example, the surface-mount, spring-loaded electronic contact structures can include pogo pins.

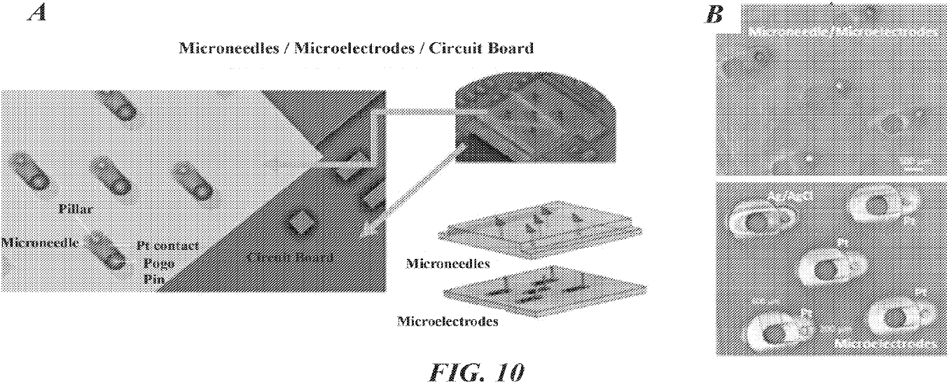

FIG. 11 shows a schematic of an example embodiment of an individual MN/MP/SP interface (panel A, left) and images of actual MN/M Pillar/Pogo-Pin connected to wires for in-vitro characterization of the sensing integration (panel B, right).

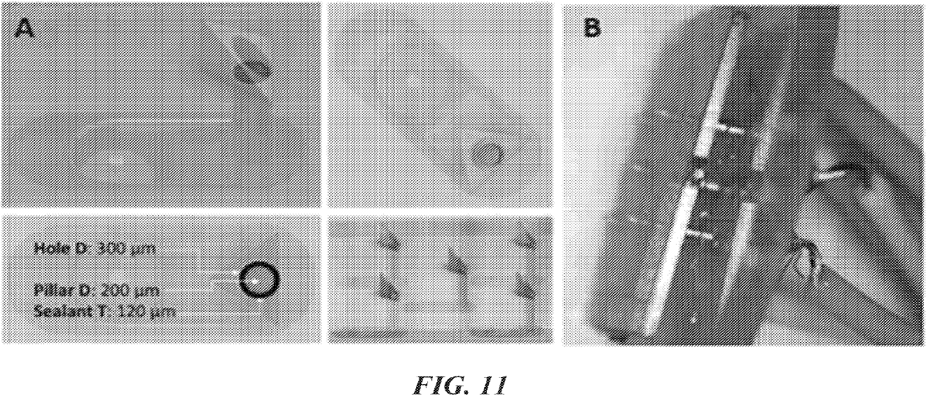

FIG. 12 shows images of an example microneedle array (panels A and B), images of example fabricated micropillars and micro-masks (panel C), and an image of an example integrated interface including MN-M-pillars and pogo-pins to the E-Board (panel D).

The disclosed technology can be used for pain-free, continuous monitoring of glucose; pain-free, continuous monitoring of other analytes individually (e.g., alcohol, lactate, etc.); pain-free, continuous monitoring of multiplex analytes subsequently using a single patch (e.g., alcohol, lactate, etc.; and controlled drug deliver patches.

The disclosed technology includes a micro-sensing interface for wearable sensing and therapeutic applications. The interface is composed of microneedle arrays which puncture the skin, micropillar microelectrodes for sensing applications, and electronics with pogo pin contacts to the micro-electrode platform. The interface allows the single use micro electro platforms to be reliably aligned and connected to the reusable electronics. The electronics are capable of testing the connections between the multi-channel and the electronics in addition to their primary function of monitoring the concentrations of various biomedically relevant analytes. The interface has been designed to support the electronics in the multi-potentiostatic control of multiple microelectrodes. The electronics allow the microelectrodes to be individually addressed and for their signals to be combined in a variety of arrangements. These features facilitate the measurement of multiple analytes using a variety of micro electro platforms. regardless of the chosen platform the interface must insure that all electrodes are connected to the electronics.

The disclosed interface has a small size, is reliable and reusable connection mechanism, and provides waterproof electronics connections. Additionally, the electronic board (E-board) is capable of multiplex sensing at the tip of microelectrodes (MN) continuously monitoring various biomarkers of interest. Moreover, for example, the fabrication method with which the microneedle/micropillars are fabricated facilitates a micromachining technique with 1 micron of true precision and reproducibility.

Example aspects of the disclosed technology include, but are not limited to: E-board-MN interface, Pillar-electrodes inserted into hollow microneedles, and sealing of the electrodes within the hollow microneedles, shown in FIGS. 10-12.

E-board-MN interface: The electronics board/microneedle integration is considered to be a critical part of the sensor integration, which is addressed in a way that is highly reproducible, scalable, and mechanically robust.

Pillar-electrodes inserted into hollow microneedles: The pillar-microneedle coupling is designed to not only be reproducible, but also provide the highest level of sensitivity and micropillar access to the ISF. The height of the micropillar electrodes are reproducibly adjustable within the microneedle hole with a desired recessed size.

Sealing of the electrodes within the hollow microneedles: Sealing of the micropillars is implemented using microtube heat-shrink insulators with desired height and reliable seal-ability making the patch to be waterproof.

The disclosed micro-interface provides a platform for wearable applications where penetration through the skin is necessary towards providing micro electrodes access to the sub-epidermal fluids such as Interstitial Fluid (ISF), and blood. Such access is done in a minimally invasive manner with negligible pain induction to the wearer while being reliably leakage proof and electronically robust. The interface can include three components: a microneedle array, micropillar microelectrodes, and electronics, e.g., depicted in FIG. 10 (panel A). The micropillar microelectrode platform can be fabricated by micromachining followed by masking and sputtering of chromium, platinum or gold, and silver at a controlled timing and sputtering pressure, e.g., shown in FIG. 10 (panel B). The silver microelectrodes can then be etched to platinum or gold and silver chloride as working and reference electrodes, respectively. Furthermore, the microelectrodes can be functionalized for continuous monitoring of variety of biomarkers through enzymatic-based detection (e.g., glucose, lactate, alcohol etc.) and/or affinity-based detection.

Further example embodiments, implementations and discussion of the disclosed microneedle sensor platform and fabrication methods are provided below.

The disclosed microneedle sensor devices, systems and platforms can include fabrication of microneedle arrays of either or both hollow and solid forms. The disclosed fabrication method provides a tremendous flexibility in fabrication of a variety of microneedles with size, shape, spacing, tip geometry, and the patch materials selection (e.g., soft metals such as aluminum and copper, machinable polymers, and some ceramics). This method is highly cost-effective, precisely reproducible (e.g., 1 µm precision), and readily scalable. Such array is applicable to many scenarios including but not limited to on-body bio-sensing applications as well as real-time extraction of human body fluids such as Interstitial Fluids (ISF) and blood, which can be used for immunosensing of important biomarkers such as testosterone, melatonin, and more. Other potential applications of the microneedles can be in epidermal drug delivery patches, and epidermal bio-fuel cells as on-body energy harvesters.

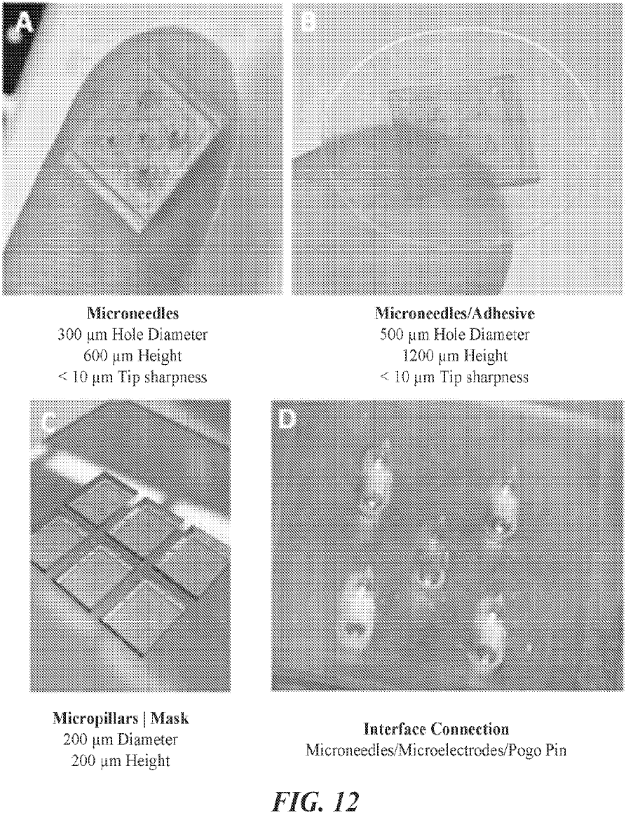

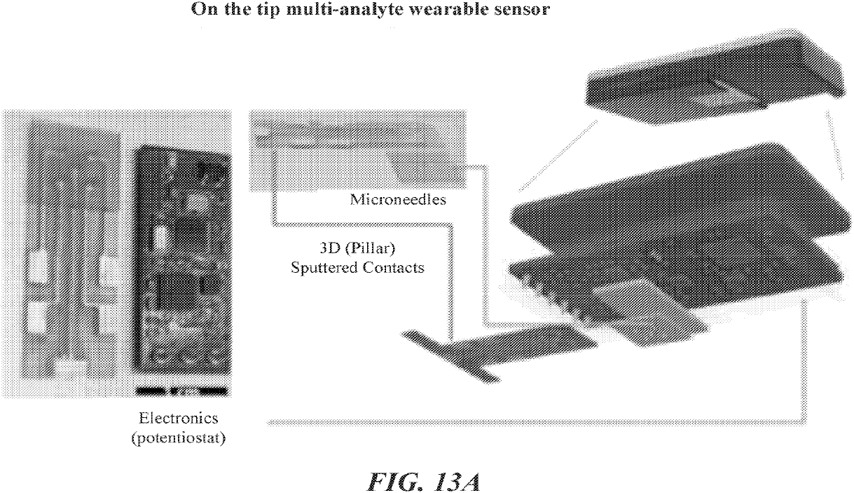

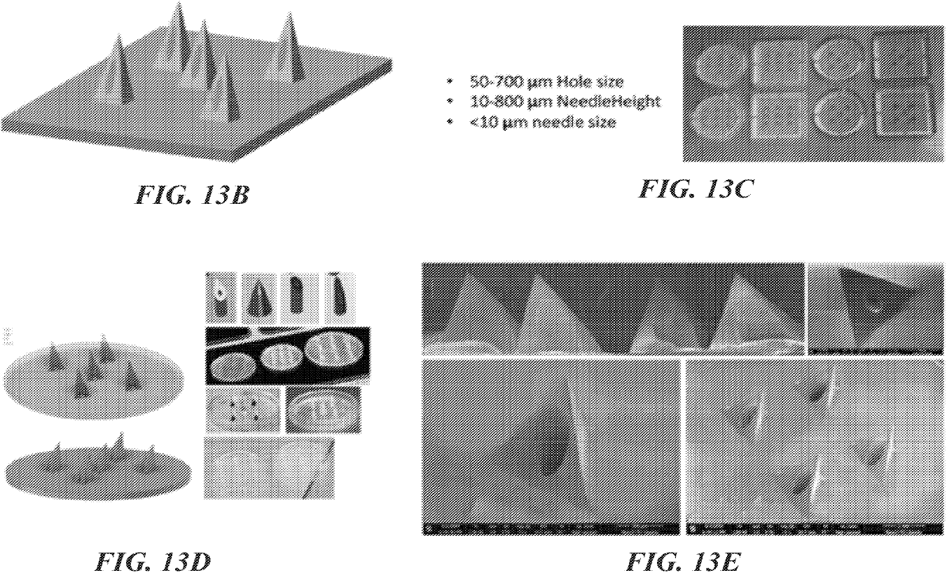

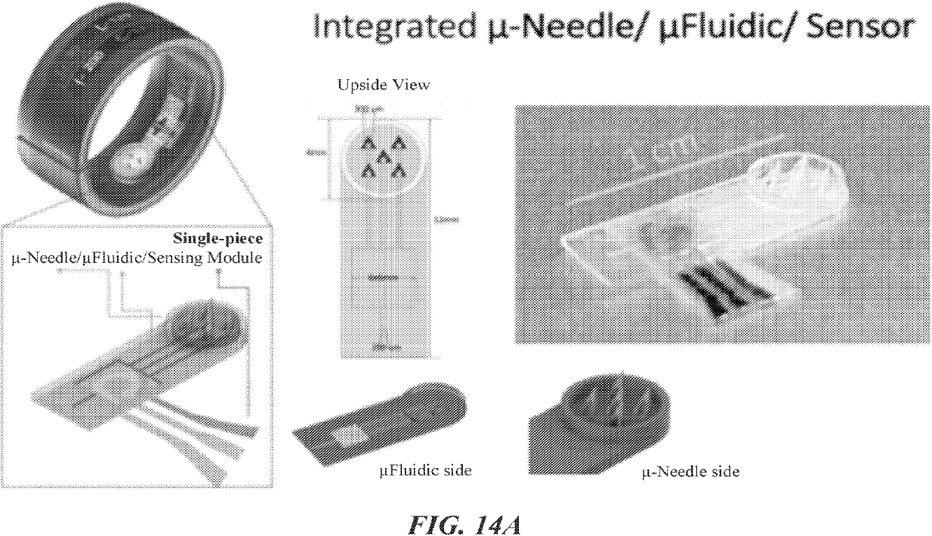

FIG. 13A shows a microneedle with micropillar electrodes connected to electronics. FIG. 13B shows a schematic of a typical microneedle patch. FIG. 13C shows microneedle arrays with variety of bases and microneedle numbers. FIG. 13D shows a 3D model of microneedles with a few fabricated arrays with different geometries. FIG. 13E shows SEMs of typical microneedle arrays.

One intention of the present technology is related to enzymatic sensing and/or immunosensing of important disease related biomarkers within human interstitial fluid (ISF). The microneedles with tip sizes down to 10 µm can be obtained which can painlessly penetrate through the skin. These microneedles have a flexible base and can be attached to the human skin tolerating relevant mechanical strains during operation. Hollow microneedles have been processed for adapting various extraction and sensing designs including “needle-tip” sensing on carbon electrodes and pillar-included microneedles of metallic gold or platinum electrodes.

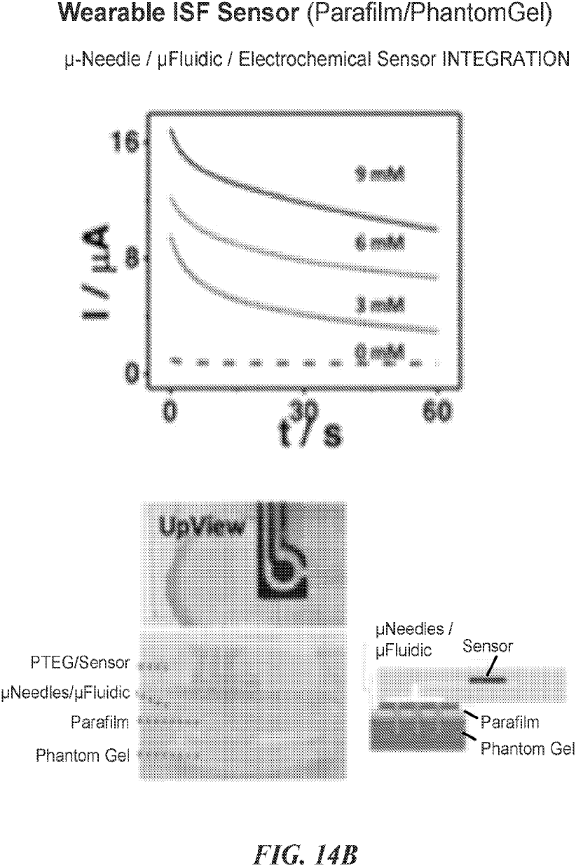

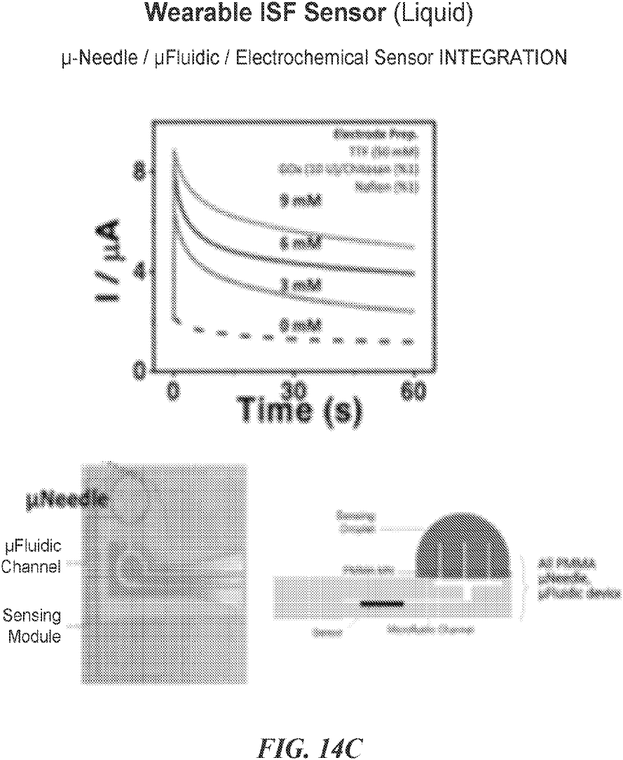

Another aspect of the present technology relates to the seamless integration of the hollow microneedle arrays with microfluidics desired for ISF extraction purposes all in the form of an integrated single device, FIG. 14A. Microfluidic channels are designed and micro-machined on the back side of the microneedle by using the same technique as the microneedles are fabricated, the micro-CNC. Workability of the microneedle-microfluidics patch has been demonstrated in-vitro by extraction and then sensing experiments as shown in FIGS. 14B and 14C.

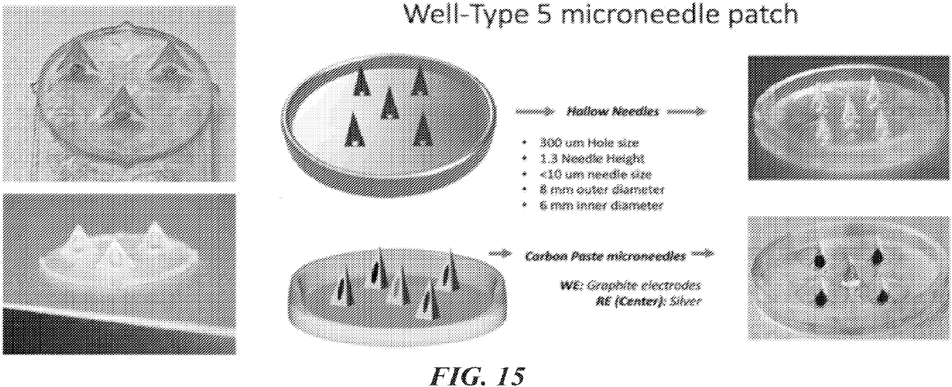

In another aspect of the present technology, described is a lithography-free method for 3D thin film sputtering of electrodes, which is used to fabricate pillar-shaped micro-electrodes, which are then integrated with microneedles (FIG. 15) towards on-the-tip sensing applications for biomarkers such as glucose, lactate, and alcohol sensing.

The disclosed microneedle fabrication method is superior to the state-of-the-art microneedle and microneedle related existing arts. This is the first example of CNC-micromachining for fabrication of microneedles. The method’s superiority is related to the design and materials selection versatility as well as its cost effectiveness over the existing art. This offers a wide range of microneedles’ geometry as well as material selection (e.g., biocompatible PEEK (Polyether ether ketone (PEEK)). Another aspect of the disclosed fabrication method relies on the microfabrication of microneedle array negative or positive molds that can be used for fabrication of hydrogel and/or PDMS microneedle arrays.

Furthermore, the aforementioned one-piece integrated microneedle-microfluidic system fabricated with this method can not only be considered as a novel device, which potentially allows extraction and sensing of ISF.

A large portion of the existing arts regarding microneedle fabrication are based on silicon materials which involves expensive and complicated microfabrication photolithography dependent methods requiring clean room facility. Due to their fragile structure, silicon microneedles are prone to fracture during transportation and clinical application. This may cause detrimental immunogenic consequences due to the unproven full bio-compatibility of silicon in case of their on-body application. Microneedles based on brittle materials such as silicon, other ceramics, and glass materials may possess similar problems. On the other hand, metallic microneedles made of metals like stainless steel, titanium, nickel, palladium, etc. have desired mechanical properties, however they suffer from several disadvantages. For example, not all the metals are biocompatible, with incompatibility towards sensing transduction strategies. In contrast, the example microneedles are based on completely biocompatible medical grade polymer materials such as Polyaryletherketone (PEEK) and medical grade polymethyl methacrylate (PMMA) or polycarbonate. Another disadvantage of silicon-based microneedles can be related to the method’s inflexibility in producing as much versatile designs.

Furthermore, the desired mechanical and electrochemical properties are achievable using the disclosed fabrication method. For instance, PMMA used in example studies has a hardness of Rockwell M94 which is comparable with Stainless Steel 316L (a biocompatible form of Stainless Steel) with a hardness of 95RB. The mechanical strength and toughness of PMMA and PEEK also are desired for reliable penetration to the skin and withstanding relevant mechanical stress and avoiding undesired fracture of the microneedles.

Another advantage of both PMMA, Acrylic, and PEEK materials, according to example experiments, is their hydrophilicity, which promotes intrinsic capillary extraction of the fluids. This is a desired property for both drug delivery and ISF/blood extraction applications. In addition, unlike other methods currently being used for polymeric microneedles fabrication like micromolding or 3D printing, the disclosed CNC fabrication method possesses a true and reproducible resolution of 1 µm and is simple and low-cost to implement.

Hollow and solid microneedle arrays can be fabricated for variety of applications from epidermal drug delivery to epidermal biosensing. The microneedles are originally designed for on-body electrochemical sensing of biomolecules such as glucose, insulin, lactate, testosterone, and more directly within the ISF or blood. At this early stage of microneedle fabrication and during consequent developments, two primary avenues were created for utilizing the disclosed microneedles. First, on-the-tip sensing, in which enzymatic working electrodes are being embedded inside and towards the tip of hollow microneedle arrays in forms of microelectrode micropillars or carbon paste fillers (FIGS. 13A and 15). Second, extraction sensing, where the detection of the biomarker of interest takes place on the sensors embedded at the back side of the microneedle array, FIGS. 14A-14C. The microneedles’ primary functionality is to pierce through the skin accessing ISF. The ISF is then extracted and directed through fluidic microchannels that are fabricated by using the same micro-CNC technique on the backside of the microneedle array.

FIG. 14A shows a single piece microneedle/microfluidic/sensor device for fluid extraction and sensing. FIG. 14B shows extraction/glucose sensing experiment results using a skin mimicking setup. FIG. 14C shows extraction/glucose sensing experiment results using a skin liquid setup.

FIGS. 14A-14C show the newly-developed single-piece integrated microfluidic-microneedle-sensor using the disclosed micro-CNC method. On-tip sensing through the fabricated microneedles is shown to be implementable in three different ways. First, by hollow microneedles being filled with carbon paste electrodes for working, counter electrodes, and silver/silver chloride paste to act as reference electrode. Second configuration is to use wire-integrated microneedles followed by subsequent functionalization of the wires which will act as the sensor electrodes. Third, and another innovative design is to integrate CNC-fabricated micropillars within hollow microneedles. These pillars can be sputtered by thin films of various metals after which upon chemical etching can form 3 electrode sensing cell without of the cumbersome photolithography-based methodology. It is believed this is the first example of using pillar shaped structures to form individually addressable electrodes and their integration with microneedles for on-body sensing applications.

The high flexibility of the micro-CNC technique to design and fabricate microneedles of various geometry and number were demonstrated. FIGS. 13C-13E show various base square or round shapes with different arrays of microneedles. These microneedles made of hard polymeric materials which can easily penetrate the human skin as shown. The performance of extraction and consequent sensing characteristics were well demonstrated by using human skin mimicking gel based on an agarose gel (%1.4 wt.) covered by Parafilm layer to simulate skin anatomy. The results obtained by an integrated microneedle-microfluidics-sensor on a skin-mimic model soaked in glucose analyte solution clearly showed the capability of the proposed design to perform ISF extraction and sensing (FIGS. 14B and 14C).

FIG. 15 shows pillar-based microneedle/microelectrodes for on the tip bio-sensing applications.

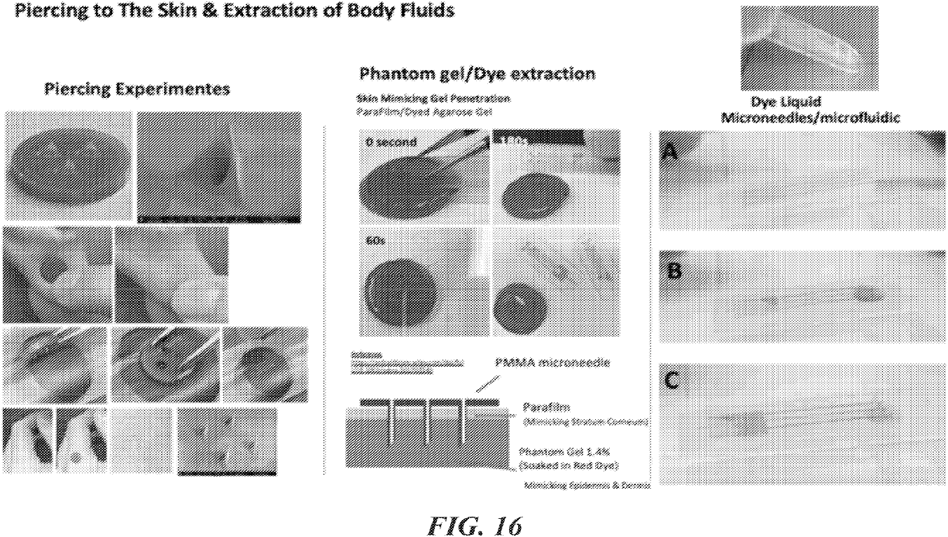

FIG. 16 shows a demonstration of the microneedle skin piercing and fluidic properties.

Example systems, devices, and methods for scalable high precision micro-fabrication of hollow and solid array microneedles with and without integrated microfluidic channels towards epidermal biosensing and drug delivery applications.

There have been a few examples of successful fabrication of hollow microneedles which are of importance to wearable sensing and drug delivery applications. Among these techniques, photolithography using silicon materials, stereolithography 3D printing techniques using photocurable resins, and injection molding techniques are the most popular. Each technique provides a specific series of advantages along with limitations, most of which are addressed using the disclosed fabrication method.

Also disclosed is a micromachining CNC method for fabrication of microneedle arrays of both hollow and solid forms. The fabrication method provides numerous advantages over its counterparts, many of which are listed as follows: (1) tremendous flexibility for manufacturing of microneedles with a variety of sizes, shapes, needles spacing, and needles tip geometry/design; (2) high flexibility in materials selection such as metals including SS316L, aluminum, copper; machinable polymers such as acrylic, polycarbonate, PEEK; Some ceramic materials; (3) high cost-effectiveness; (4) precisely reproducible (e.g., 1 µm precision), a true resolution up to 1 µm; and (5) high scalability, e.g., the fastest manufacturing process with high yield. (e.g., 20 high-quality 10 by 10 MNs patches in <5 minutes).

Summary of the advantages of the CNC fabrication method over conventional techniques is listed in Table 1.

TABLE 1

Qualitative comparison of disclosed micro-CNC fabrication method with conventional methods for fabrication of the hollow and solid microneedles

| | | | | |
|---|---|---|---|---|
| | Micro-CNC | Highest end 3D printers | Injection molding | Photolithography based |
| Capital cost | $20k for the machine | $10k for the machine | $20k for the system | >$500k for the facility |
| Materials costs/MN-patch | 0.01-0.5$/microneedle patch (varies by stock materials) | $2-10 depending upon the resin | low | >$10 for high thickness silicon wafers |
| Resolution (X-Y-Z) | 1 µm XYZ | 25 µm XYZ | 50 µm XYZ | 1 µm XYZ |
| Tip sharpness | <10 µm | >25 µm | >25 µm | |
| Hollow microneedles | YES, 50 µm hole size limit | YES/400 µm hole size limit | YES, 400 µm hole size limit | YES, 50 µm hole size limit |
| Geometry | Highly Versatile | Highly versatile | versatile | limited height and geometry/shape |
| Materials | All machinable Polymers/metals like Al, Cu,316LSS/machinable ceramics | Photo curable Polymers | Some Polymers | Si, Sio2 |
| Biocompatibility | YES (PEEK, Acrylic, SS316L, and more) | YES | YES | YES |
| Mechanical properties | Flexible, Stiff enough for piercing the skin | Flexible, Stiff enough for piercing the skin | Flexible, NOT stiff enough for piercing the skin | Rigid, Stiff enough for piercing the skin |
| Scalability | YES, Fastest in industry, High yield, highly Reproducible | YES, much slower yield. Reproducible | YES, faster, reproducible, | YES, faster, reproducible. |
| Reproducibility | YES, <10 µm tolerance | YES, <25 µm tolerance | YES, <50 µm tolerance | YES, <1 µm tolerance |
| Surface roughness | <1 µm | <25 µm | <50 µm | <1 µm |

IDEAL (in bold)
BELOW IDEAL (underlined)

### Aspect 1: Hollow Microneedles

To fabricate a microneedle array, a design of the array was first produced in a computer aided design and modeling software such as Fusion 360 and/or AutoCAD. The designed models are then decoded by a computer-aided manufacturing using a unique combination and sequence of tool and drill bits. Depending upon the desired shape and design of the microneedles, a two flute bit with diameter sizes ranging from 100 µm-1.2 mm is used to engrave the outline structure of the microneedles in series. The cutting strategy (e.g., 2D pocket vs. radial and trace) with specific combination of cutting parameters (e.g., spindle speed of 12000-17000 rpm, bit moving speed ranging from 400-1500 mm/min, and cutting step size ranging from 0.2-0.7 mm) are used in accordance to the final microneedle size, for example.

Drilling bits ranging from 50 µm-800 µm are used to create holes using a spindle speed ranging from 25000-27000 rpm, and drilling strategy of either rapid drilling for microneedles with holes bellow 300 µm or strategy pecking depth drilling for microneedles with holes above 300 µm. Lastly, a roughing step is implemented using round ball end mills with diameters ranging from 200 µm-800 µm, creating an extremely smooth surface for the needles.

Furthermore, the manufacturing rate of the hollow microneedles can be significantly shifted by employing combination of tracing strategy and groove end mills. Such strategy, while providing aforementioned advantages, allows for high rate mass-producibility. To obtain microneedle arrays using trace strategy, first, micro-holes are drilled to the stock, then tracing takes place using bits with desired tip angles (e.g., 10 to 45 degrees). The height, spacing and number of microneedle arrays are controlled by tracing spacing, the choice of bit geometry, and post processing by flat end mills with diameters ranging from 100 µm to 1.2 mm, respectively.

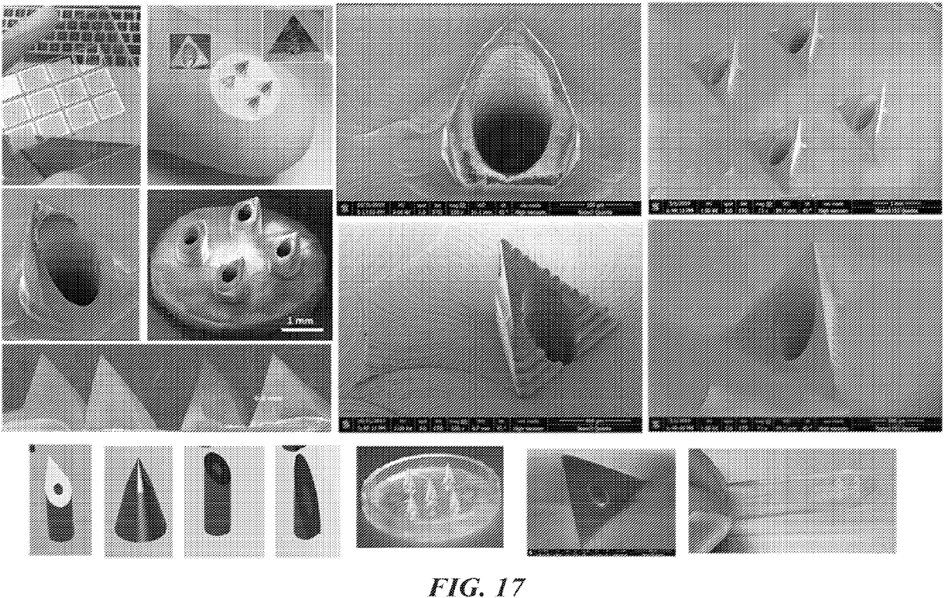

FIG. 17 shows photographs and SEM images of microneedles with variety of shapes, sizes and spacing demonstrating the high precision and resolution of the micro-CNC machining technique.

### Example Advantages

As outlined in the Table 1, there are multiple advantages to the CNC micromachining fabrication method, some of which include high reproducibility, precision, true resolution of 1 µm, and mass-producibility. These allow fabrication of microneedles with sharp edges and bellow 10 µm tip size, FIG. 17, with desired height, and needle geometry.

Such level of precision is found only in photolithography-based method. However, common multistep photolithographic methods, which require a clean room facility, are limited in regards to the geometry and height of the microneedles fabricated as well as the materials selectivity being constrained to silicon wafers which are usually commercially available in thicknesses bellow 800 µm.

### Aspect 2: Solid Microneedles

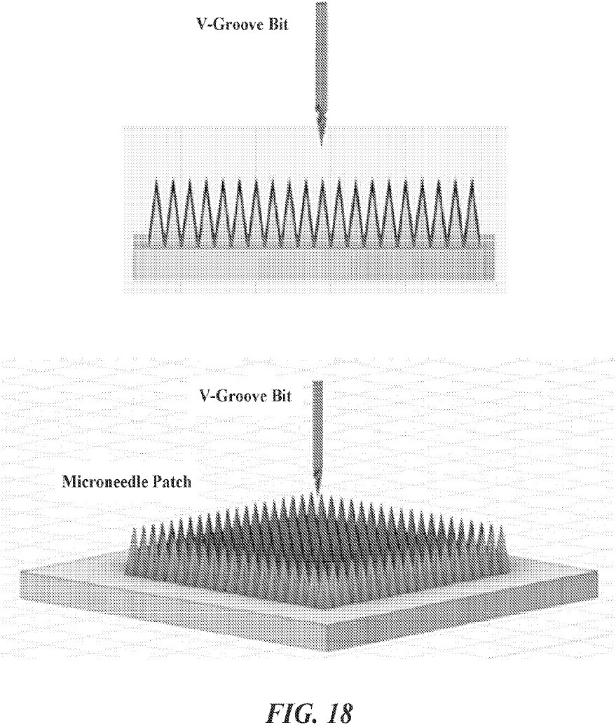

Aside from fabrication of hollow microneedles using the disclosed techniques, a method for rapid fabrication of solid microneedles with extremely sharp tips ideal for on-the-tip wearable sensing applications as well as drug delivery. Particular CNC V-groove bits, FIG. 18, are used combined with trace strategy in 4 straight line directions, which leaves formation of multiple microneedles in a matter of seconds (e.g., a 20-20 microneedle array fabricated within 50 seconds). The V-groove routing it angle ranges from 10-60 degrees with specific penetration to the stock material ranging from 400-1200 µm, which reflects the ultimate height of the microneedles. The cutting strategy chose is tracing with specific combination of cutting parameters (e.g, spindle speed of 12000-17000 rpm, bit moving speed ranging from 400-1500 mm/min, and cutting step size ranging from 0.2-0.7 mm) are used in accordance to the final microneedle as well as the bit diameter used.

The primary advantage of this method is regarding the speed with which the microneedle patches are being fabricated, the range of height, as well as all the aforementioned advantages of the developed micro-CNC technique.

FIG. 18 shows a solid microneedle patch using V-Groove and Trace strategy.

### Aspect 3: Single-Pece Integrated Microneedles/Microfluidic Channels Device

An integrated single-piece microneedle/microfluidic (MN/MF) channel device is extremely important for piercing the skin followed by controlled ISF/Blood extraction by microfluidic channels that seamlessly transfer the fluid to the sensing chamber without causing any evaporation of the fluid or/and contamination by sweat, and potential skin products. Another aspect of the disclosed technology relates to the fabrication of hollow microneedles with desired geometry, height and hole size on one side of the stock and microfluidic channels extending the microneedle holes to channels which can be used for extraction of body fluids of interest for sensing applications or injection of drugs from the microfluidic chamber/channels to within the skin for drug delivery applications.

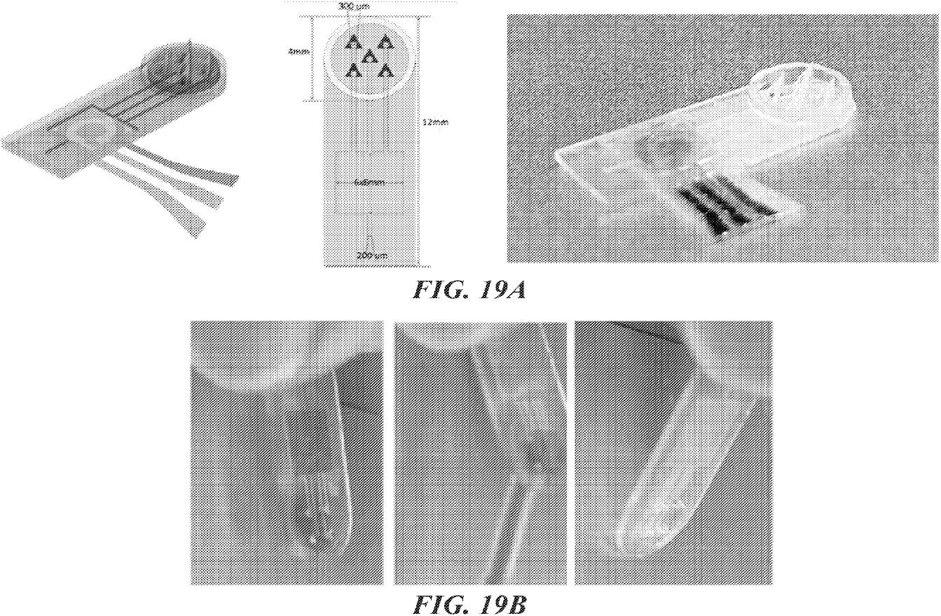

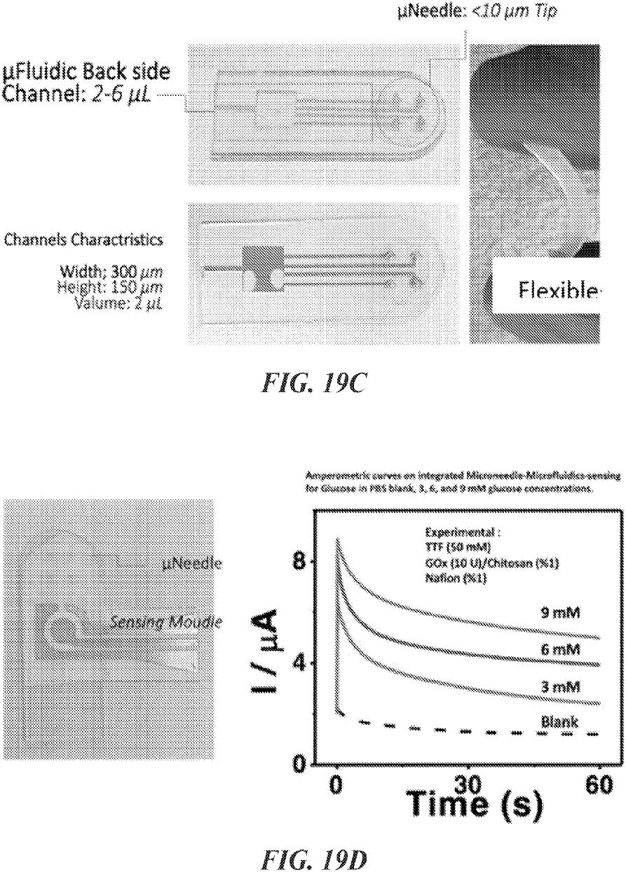

FIGS. 19A-19D show diagrams, images and example results associated with example implementations of an example single-piece microneedle/microfluidic channel device with sensor unit integration, in accordance with the present technology. FIG. 19A shows diagrams and an image of the example single-piece microneedle/microfluidic channel device with sensor unit integration. FIG. 19B shows an extraction of dyed fluid. FIG. 19C shows a demonstration of fluid extraction from the needles’ holes through the microfluidic channels by capillary forces. Flexible piece demonstrated. FIG. 19D shows in-vitro glucose extraction/sensing.

To fabricate the single piece integrated microneedles/microfluidic channels device, a similar combination of micro CNC milling strategy/ bit tool, as described in the hollow microneedle section, is used for fabrication of the hollow microneedles with hole sizes bellow 100 microns on one side of the stock material. Then, a specific mirroring alignment technique is used to form microfluidic channels on the opposite side of the stock material with the channels being precisely aligned on the microneedle holes. The alignment step include formation of 4 holes on the microneedle side which are then used as the zeroing point when turning the stuck over to fabrication the microchannels.

A primary advantage of the disclosed method over other competing fabrication techniques (i.e., high precision 3D printing) relates to the miniaturization and the size range (bellow 100 micron) of the microneedle/microfluidic channels with a true precision of 1 micron, which is of importance for precise control over the volume of the fluidic that is either extracted (e.g., ISF with reported extraction of bellow 20 µL, and blood for sensing) or injected (e.g., drugs in fluid form).

Furthermore, the other advantage of the disclosed method relates to the mechanical flexibility of the single piece which could be of importance for on-body applications, as depicted in FIGS. 19A-19D. The mechanical flexibility of the single-piece device is not an advantage that is not available for silicon and glass-based microneedle microfluidic systems. Additionally, PDMS based microfluidic devices, as a counterpart to the disclosed device, lack microneedle array component and in most cases they suffer from fluid leakage problem, which stems from low bonding ability of the PDMS. This is while, the example single-piece device in accordance with the disclosed technology can reliability be sealed almost any adhesive films, e.g., such as a simple scotch tape.

Furthermore, as shown in FIGS. 19B and 19C, the inherent hydrophilicity of the acrylic materials promotes spontaneous capillary suction of the fluids (e.g., ISF, or blood). Such property combined with the micron range size of the MN/MF single piece device with hole sizes of 50-100 µm and channel size of 50-100 µm with multiple needles with a tip size bellow 10 µm circumference in a well like structure can be considered as another novelty point of the device. The example device not only combines both sensing and extraction capabilities but it also provides such functionalities in a miniaturized wearable form factor, which is scalable and inexpensively reproducible.

### Examples

In some embodiments in accordance with the present technology (example 1), a wearable medical device for simultaneous and continuous monitoring of a plurality of analytes includes (a) a microneedle array sensor unit, comprising (i) an array of microneedles, each comprising an exterior wall forming a protruding needle structure converging at an apex point, wherein each microneedle includes a hollow interior defined by an interior wall, and an opening disposed on the exterior wall leading to the hollow interior, and (ii) an array of micropillar electrodes, each disposed within the hollow interior of a respective protruding needle structure, wherein a first micropillar electrode of the array is configured to interact with a first analyte that comes in contact with a first sensing region of the first micropillar electrode to produce a first electrical signal indicative of an electrochemical reaction involving the first analyte at the first sensing region, and wherein a second micropillar electrode of the array is configured to interact with a second analyte that comes in contact with a second sensing region of the second micropillar electrode to produce a second electrical signal indicative of an electrochemical reaction involving the second analyte at the second sensing region; (b) an electronics unit in electrical communication with the array of micropillar electrodes, the electronics unit comprising a power source, a signal processing circuit, and a wireless transmitter; (c) a plurality of surface-mount, spring-loaded pins that electrically couple the micropillar electrodes of the array to the signal processing circuit of the electronics unit; and (d) a housing structure to encase the electronics unit and to encase, at least partially, the microneedle array sensor unit, such that at least the apex point and the opening of each microneedle of the array of microneedles protrude outward of the housing structure, wherein the array of microneedles is exposed from a side of the housing structure.

Example 2 includes the device of example 1 or examples 3-15, wherein the electronics unit includes a two-sided printed circuit board (PCB) to integrate electronic components including at least the signal amplification circuit, the wireless transmitter, and the power source, wherein a one side of the two-sided PCB includes the plurality of surface-mount, spring-loaded pins that electrically couple the micropillar electrodes to the signal amplification circuit and/or the power supply, such that at least one of the surface-mount, spring-loaded pins is configured to create an automatic power switch of the wearable medical device, the automatic power switch providing an electrical conduction path through the array of microneedles to be on when the array of micropillar electrodes are inserted within the array of microneedles, and wherein the electrical conduction path is broken when the micropillar electrodes are not inserted within the array of microneedles.

Example 3 includes the device of any of examples 1-2 or examples 4-15, wherein the protruding needle structure of at least some of the microneedles of the array includes one or more of (i) one exterior wall such that the protruding needle structure is of a conical shape, (ii) three exterior walls such that the protruding needle structure is of a triangular pyramid shape, or (iv) four exterior walls such that the protruding needle structure is of a rectangular pyramid shape.

Example 4 includes the device of any of examples 1-3 or examples 7-15, wherein the array of micropillar electrodes includes a first electrode probe structure including a first sensing layer comprising a first biological or chemical substance configured to facilitate the electrochemical reaction with the first analyte, and wherein the array of micropillar electrodes includes a second electrode probe structure includes a second sensing layer comprising a second biological or chemical substance configured to facilitate the electrochemical reaction with the second analyte.

Example 5 includes the device of example 4, wherein the array of micropillar electrodes includes a third electrode probe structure configured as a counter electrode to detect a signal using an electrochemical detection technique with respect to that detected by the first electrode probe structure and the second electrode probe structure.

Example 6 includes the device of example 5, wherein the array of micropillar electrodes includes a fourth electrode probe structure configured as a reference electrode to detect a signal using an electrochemical detection technique with respect to that detected by the first electrode probe structure and the second electrode probe structure.

Example 7 includes the device of any of examples 1-6 or examples 8-15, wherein the microneedles of the array of microneedles include a height ranging from 400 µm-800 µm.

Example 8 includes the device of any of examples 1-7 or examples 9-15, wherein the electrochemical detection technique includes one of potentiometry, cyclic voltammetry (CV), fast scan cyclic voltammetry (FSCV), square wave voltammetry (SWV), or chronoamperometry.

Example 9 includes the device of any of examples 1-8 or examples 11-15, further comprising a computer program product executable as a software application, resident on a mobile communication device in communication with the electronics unit, wherein the computer program product is able to control one or more of (i) detection of electrochemical measurements conducted at the microneedle array sensor unit, (ii) data analysis, (iii) data transmission, or (iv) device power management.

Example 10 includes the device of example 9, wherein the mobile communication device includes a smartphone, a smartwatch, a tablet, a smartglasses, a laptop, or one or more computers in the cloud.

Example 11 includes the device of any of examples 1-10 or examples 14-15, wherein the plurality of analytes includes a diabetes biomarker including two or more of glucose, lactate, alcohol, or an electrolyte.

Example 12 includes the device of any of examples 1-11 or example 13, wherein one or more of the analytes are contained in a fluid deposited on the microneedle array sensor unit, the fluid including saliva, blood, tears, interstitial fluid, or combination thereof.

Example 13 includes the device of any of examples 1-12, wherein the device can be configured as a patch worn on skin of a patient user.

In some embodiments in accordance with the present technology (example 14), a method for simultaneous and continuous monitoring of a plurality of analytes in a fluid of a patient user in a non-invasive fashion, the method comprising: disposing the device of any of Examples 1-13, 15-22, 23-36, or 37-53 on a skin of the patient user; detecting at least the first electrical signal and the second electrical signal produced by the device; and wirelessly transmitting detected electrical signals from the device to a mobile communication device.

In some embodiments in accordance with the present technology (example 15), a wearable medical device for collection of interstitial fluid (ISF) includes (a) a device body including a chamber enclosing an internal volume, the device body having a first side and a second side, wherein the first side of the device body is opposite to the second side of the device body; (b) an array of microneedles protruding from the first side of the device body, the array of microneedles operable to pierce skin when the first side of the device body is at or proximate to contact with the skin, wherein each microneedle in the array of microneedles comprises at least one exterior wall forming a protruding needle structure converging at an apex point, each microneedle having a hollow interior region defined by an interior wall that leads to an exterior opening located on the at least one exterior wall of the protruding needle structure, and wherein the hollow interior region of each microneedle in the array of microneedles is fluidically coupled to the internal volume of the device body through a first opening on the first side of the device body; and (c) a suction assembly disposed at the second side of the device body and fluidically coupled to the internal volume of the chamber through a second opening in the second side.

Example 16 includes the device of example 15 wherein the suction assembly comprises a fluidic channel capable of suction via capillary force, or a pump, or both the fluidic channel and the pump, fluidically coupled to the internal volume of the device body through the second opening on the second side of the device body.

Example 17 includes the device of example 16, wherein the second side of the device body comprises a pump port having a first end, a second end, a side surface enclosing a hollow internal volume of the pump port between the first end and the second end and having a first pump port opening into the internal volume of the pump port at the first end and a second pump port opening into the internal volume of the pump port at the second end, wherein the pump port is connected to the second side of the device body at the first end of the pump port such that the internal volume of the pump port is fluidically coupled to the internal volume of the device body through the first pump port opening into the internal volume of the pump port at the first end and the second opening on the second side of the device body.

Example 18 includes the device of example 16, wherein the pump is connected to the pump port using a tubing connected to the second end of the pump port.

Example 19 includes the device of example 15, wherein the suction assembly comprises a bellow having a first bellow end, a second bellow end, a side surface enclosing a hollow internal volume of the bellow between the first bellow end and the second bellow end and having a first bellow opening into the internal volume of the bellow at the first bellow end; a spring having a first spring end and a second spring end and positioned inside the bellow such that the first spring end is proximate to the first bellow end and the second spring end is proximate to the second bellow end, the spring is coiled around an axis connecting the first bellow end and the second bellow end, the spring contacts the device body at the first spring end, the spring contacts the bellow at the second spring end, and the spring is configured to contract and/or expand along the axis connecting the first bellow end and the second bellow end, wherein: the bellow is attached to the second side of the device body at the first bellow end such that the internal volume of the bellow is fluidically coupled to the internal volume of the device body through the first bellow opening into the internal volume of the bellow at the first bellow end and the second opening on the second side of the device body; and the bellow is configured to transition between a contracted state and an expanded state wherein a distance between the first bellow end and the second bellow end in the expanded state is larger than the distance between the first bellow end and the second bellow end in the contracted state and wherein a transition of the bellow between the contracted state and the expanded state leads to an increase of the internal volume of the bellow.

Example 20 includes the device of any of examples 15-19, wherein the array of microneedles includes a microneedle array sensor unit, comprising: an array of micropillar electrodes, each disposed within the hollow interior region of a respective protruding needle structure of the array of microneedles, wherein a micropillar electrode of the array of micropillar electrodes is configured to interact with an analyte that comes in contact with a sensing region of the micropillar electrode via the exterior opening of the microneedle to produce an electrical signal indicative of an electrochemical reaction involving the analyte.

Example 21 includes the device of example 20, wherein the array of micropillar electrodes include a first electrode of the array configured to interact with a first analyte that interacts with the first electrode to produce a first electrical signal indicative of an electrochemical reaction involving the first analyte, and wherein a second electrode of the array is configured to interact with a second analyte that interacts with the second electrode to produce a second electrical signal indicative of an electrochemical reaction involving the second analyte.

Example 22 includes the device of any of examples 20-21, wherein the device is configured to include or interface with an electronics unit to be in electrical communication with the array of micropillar electrodes, the electronics unit comprising a power source, a data processing unit, and a wireless transmitter, wherein the device includes a plurality of surface-mount, spring-loaded pins that electrically couple the micropillar electrodes of the array to the signal processing circuit of the electronics unit.

In some embodiments in accordance with the present technology (example 23), a wearable microneedle sensor device for continuous analyte monitoring includes an array of microneedles, each comprising an exterior wall forming a protruding needle structure converging at an apex point, wherein each microneedle includes a hollow interior defined by an interior wall, and an opening disposed on the exterior wall leading to the hollow interior; an array of micropillar electrodes, each disposed within the hollow interior of a respective protruding needle structure, wherein the micropillar electrodes of the array are configured to interact with an analyte that comes in contact with a sensing region of a respective micropillar electrode to produce an electrical signal indicative of an electrochemical reaction involving the analyte at the sensing region; an electronics unit in electrical communication with the array of micropillar electrodes, the electronics unit comprising a power source, a signal processing circuit, and a wireless transmitter; a plurality of surface-mount, spring-loaded pins that electrically couple the micropillar electrodes of the array to the signal processing circuit of the electronics unit; and a housing structure to encase the electronics unit and to encase, at least partially, the array of microneedles and array of micropillar electrodes, such that at least the apex point and the opening of each microneedle of the array of microneedles protrude outward of the housing structure, wherein the array of microneedles is exposed from a side of the housing structure.

Example 24 includes the device of example 23, wherein the electronics unit includes a two-sided printed circuit board (PCB) to integrate electronic components including at least the signal amplification circuit, the wireless transmitter, and the power source, wherein a one side of the two-sided PCB includes the plurality of surface-mount, spring-loaded pins that electrically couple the micropillar electrodes to the signal amplification circuit and/or the power supply.

Example 25 includes the device of example 24, wherein the device includes an automatic power switch comprising at least one of the surface-mount, spring-loaded pins, wherein the automatic power switch is configured to provide an electrical conduction path through the array of microneedles in an on state when the array of micropillar electrodes are inserted within the array of microneedles, and wherein the electrical conduction path is broken when the micropillar electrodes are not inserted within the array of microneedles such that the automatic power switch is in an off state.

Example 26 includes the device of any of examples 23-25, wherein the protruding needle structure of at least some of the microneedles of the array includes one or more of (i) one exterior wall such that the protruding needle structure is of a conical shape, (ii) three exterior walls such that the protruding needle structure is of a triangular pyramid shape, or (iv) four exterior walls such that the protruding needle structure is of a rectangular pyramid shape.

Example 27 includes the device of any of examples 23-26 or examples 30-36, wherein the array of micropillar electrodes includes a first electrode probe structure including a first sensing layer comprising a first biological or chemical substance configured to facilitate the electrochemical reaction with the first analyte, and wherein the array of micropillar electrodes includes a second electrode probe structure includes a second sensing layer comprising a second biological or chemical substance configured to facilitate the electrochemical reaction with the second analyte.

Example 28 includes the device of example 27, wherein the array of micropillar electrodes includes a third electrode probe structure configured as a counter electrode to detect a signal using an electrochemical detection technique with respect to that detected by the first electrode probe structure and the second electrode probe structure.

Example 29 includes the device of example 28, wherein the array of micropillar electrodes includes a fourth electrode probe structure configured as a reference electrode to detect a signal using an electrochemical detection technique with respect to that detected by the first electrode probe structure and the second electrode probe structure.

Example 30 includes the device of any of examples 23-29 or examples 31-36, wherein the microneedles of the array of microneedles include a height ranging from 400 µm-800 µm.

Example 31 includes the device of any of examples 23-30 or examples 32-36, wherein the electrochemical detection technique includes one of potentiometry, cyclic voltammetry (CV), fast scan cyclic voltammetry (FSCV), square wave voltammetry (SWV), or chronoamperometry.

Example 32 includes the device of any of examples 23-31 or examples 33-36, further comprising a computer program product executable as a software application, resident on a mobile communication device in communication with the electronics unit, wherein the computer program product is able to control one or more of (i) detection of electrochemical measurements conducted at the microneedle array sensor unit, (ii) data analysis, (iii) data transmission, or (iv) device power management.

Example 33 includes the device of example 32, wherein the mobile communication device includes a smartphone, a smartwatch, a tablet, a smartglasses, a laptop, or one or more computers in the cloud.

Example 34 includes the device of any of examples 23-33 or examples 34-36, wherein the plurality of analytes includes a diabetes biomarker including two or more of glucose, lactate, alcohol, or an electrolyte.

Example 35 includes the device of any of examples 23-34 or example 36, wherein one or more of the analytes are contained in a fluid deposited on the microneedle array sensor unit, the fluid including saliva, blood, tears, interstitial fluid, or combination thereof.

Example 36 includes the device of any of examples 23-35, wherein the device can be configured as a patch worn on skin of a patient user.

In some embodiments in accordance with the present technology (example 37), a wearable microneedle sensor device for continuous analyte monitoring, comprising (a) a flexible, electrically-insulative substrate; (b) an array of microneedles disposed at a first region of the substrate, each microneedle of the array comprising an exterior wall forming a protruding needle structure converging at an apex point, wherein each microneedle includes a hollow interior defined by an interior wall, and an opening disposed on the exterior wall leading to the hollow interior; (c) an array of electrodes disposed at a second region of the substrate, wherein the second region includes a cavity having a bottom surface on which the electrodes of the array are positioned, wherein the electrodes includes a working electrode and at least one of a reference electrode or counter electrode, and wherein the array of electrodes are configured to detect an analyte in a fluid that is provided within the cavity such that the electrodes are operable to produce an electrical signal indicative of an electrochemical reaction involving the analyte at the working electrode; (d) a plurality of microfluidic channels disposed in the substrate between the cavity and the hollow interior of each microneedle of the array of microneedles, respectively; (e) an array of electrode interface terminals that are coupled to the array of electrodes, respectively, wherein each of the array of electrode interface terminals includes a contact region positioned away from a region that is coupled to the electrode, respectively; and (f) an electronics unit in electrical communication with the array of electrode interface terminals, the electronics unit comprising a power source, a signal processing circuit, and a wireless transmitter.

Example 38 includes the device of any of example 37 or examples 41-53, further comprising a housing structure to encase the electronics unit and to encase, at least partially, the microneedle array sensor unit, such that at least the apex point and the opening of each microneedle of the array of microneedles protrude outward of the housing structure, wherein the array of microneedles is exposed from a side of the housing structure.

Example 39 device of example 38, wherein the housing structure is configured as a band that is wearable on an appendage of a patient user, such that the apex point of the microneedle is disposed on an inside face of the band.

Example 40 includes the device of example 39, wherein the band includes a digital display on an outside face of the band.

Example 41 includes the device of any of examples 37-40 or examples 42-53, further comprising: a plurality of surface-mount, spring-loaded pins that electrically couple the contact region of each of the electrode interface terminals of the array to the signal processing circuit of the electronics unit.

Example 42 includes the device of example 41, wherein the electronics unit includes a two-sided printed circuit board (PCB) to integrate electronic components including at least the signal amplification circuit, the wireless transmitter, and the power source, wherein a one side of the two-sided PCB includes the plurality of surface-mount, spring-loaded pins that electrically couple the micropillar electrodes to the signal amplification circuit and/or the power supply.

Example 43 includes the device of any of examples 37-42 or examples 44-53, wherein the protruding needle structure of at least some of the microneedles of the array includes one or more of (i) one exterior wall such that the protruding needle structure is of a conical shape, (ii) three exterior walls such that the protruding needle structure is of a triangular pyramid shape, or (iv) four exterior walls such that the protruding needle structure is of a rectangular pyramid shape.

Example 44 includes the device of any of examples 37-43 or examples 45-53, wherein the array of micropillar electrodes includes a first electrode probe structure including a first sensing layer comprising a first biological or chemical substance configured to facilitate the electrochemical reaction with the first analyte, and wherein the array of micropillar electrodes includes a second electrode probe structure includes a second sensing layer comprising a second biological or chemical substance configured to facilitate the electrochemical reaction with the second analyte.

Example 45 includes the device of example 44, wherein the array of micropillar electrodes includes a third electrode probe structure configured as a counter electrode to detect a signal using an electrochemical detection technique with respect to that detected by the first electrode probe structure and the second electrode probe structure.

Example 46 includes the device of example 45, wherein the array of micropillar electrodes includes a fourth electrode probe structure configured as a reference electrode to detect a signal using an electrochemical detection technique with respect to that detected by the first electrode probe structure and the second electrode probe structure.

Example 47 includes the device of any of examples 37-46 or examples 48-53, wherein the microneedles of the array of microneedles include a height ranging from 400 µm-800 µm.

Example 48 includes the device of any of examples 37-47 or examples 49-53, wherein the electrochemical detection technique includes one of potentiometry, cyclic voltammetry (CV), fast scan cyclic voltammetry (FSCV), square wave voltammetry (SWV), or chronoamperometry.

Example 49 includes the device of any of examples 37-48 or examples 50-53, further comprising a computer program product executable as a software application, resident on a mobile communication device in communication with the electronics unit, wherein the computer program product is able to control one or more of (i) detection of electrochemical measurements conducted at the microneedle array sensor unit, (ii) data analysis, (iii) data transmission, or (iv) device power management.

Example 50 includes the device of example 49, wherein the mobile communication device includes a smartphone, a smartwatch, a tablet, a smartglasses, a laptop, or one or more computers in the cloud.

Example 51 includes the device of any of examples 37-50 or examples 52-53, wherein the plurality of analytes includes a diabetes biomarker including two or more of glucose, lactate, alcohol, or an electrolyte.

Example 52 includes the device of any of examples 37-51 or example 53, wherein one or more of the analytes are contained in a fluid deposited on the microneedle array sensor unit, the fluid including saliva, blood, tears, interstitial fluid, or combination thereof.

Example 53 includes the device of any of examples 37-52, wherein the device can be configured as a patch worn on skin of a patient user.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

1. A wearable medical device for simultaneous and continuous monitoring of a plurality of analytes, comprising:
a microneedle array sensor unit, comprising:
an array of microneedles, each comprising an exterior wall forming a protruding needle structure converging at an apex point, wherein each microneedle includes a hollow interior defined by an interior wall, and an opening disposed on the exterior wall leading to the hollow interior, and
an array of micropillar electrodes, each disposed within the hollow interior of a respective protruding needle structure, wherein a first micropillar electrode of the array is configured to interact with a first analyte that comes in contact with a first sensing region of the first micropillar electrode to produce a first electrical signal indicative of an electrochemical reaction involving the first analyte at the first sensing region, and wherein a second micropillar electrode of the array is configured to interact with a second analyte that comes in contact with a second sensing region of the second micropillar electrode to produce a second electrical signal indicative of an electrochemical reaction involving the second analyte at the second sensing region;
an electronics unit in electrical communication with the array of micropillar electrodes, the electronics unit comprising a power source, a signal processing circuit, and a wireless transmitter;
a plurality of surface-mount, spring-loaded pins that electrically couple the micropillar electrodes of the array to the signal processing circuit of the electronics unit; and
a housing structure to encase the electronics unit and to encase, at least partially, the microneedle array sensor unit, such that at least the apex point and the opening of each microneedle of the array of microneedles protrude outward of the housing structure, wherein the array of microneedles is exposed from a side of the housing structure.

2. The device of claim 1, wherein the electronics unit includes a two-sided printed circuit board (PCB) to integrate electronic components including at least the signal amplification circuit, the wireless transmitter, and the power source, wherein a one side of the two-sided PCB includes the plurality of surface-mount, spring-loaded pins that electrically couple the micropillar electrodes to the signal amplification circuit and/or the power supply, such that at least one of the surface-mount, spring-loaded pins is configured to create an automatic power switch of the wearable medical device, the automatic power switch providing an electrical conduction path through the array of microneedles to be on when the array of micropillar electrodes are inserted within the array of microneedles, and wherein the electrical conduction path is broken when the micropillar electrodes are not inserted within the array of microneedles.

3. The device of claim 1, wherein the protruding needle structure of at least some of the microneedles of the array includes one or more of (i) one exterior wall such that the protruding needle structure is of a conical shape, (ii) three exterior walls such that the protruding needle structure is of a triangular pyramid shape, or (iv) four exterior walls such that the protruding needle structure is of a rectangular pyramid shape.

4. The device of claim 1, wherein the array of micropillar electrodes includes a first electrode probe structure including a first sensing layer comprising a first biological or chemical substance configured to facilitate the electrochemical reaction with the first analyte, and wherein the array of micropillar electrodes includes a second electrode probe structure includes a second sensing layer comprising a second biological or chemical substance configured to facilitate the electrochemical reaction with the second analyte.

5. The device of claim 4, wherein the array of micropillar electrodes includes a third electrode probe structure configured as a counter electrode to detect a signal using an electrochemical detection technique with respect to that detected by the first electrode probe structure and the second electrode probe structure.

6. The device of claim 5, wherein the array of micropillar electrodes includes a fourth electrode probe structure configured as a reference electrode to detect a signal using an electrochemical detection technique with respect to that detected by the first electrode probe structure and the second electrode probe structure.

7. The device of claim 1, wherein the microneedles of the array of microneedles include a height ranging from 400 µm-800 µm.

8. The device of claim 1, wherein the electrochemical detection technique includes one of potentiometry, cyclic voltammetry (CV), fast scan cyclic voltammetry (FSCV), square wave voltammetry (SWV), or chronoamperometry.

9. The device of claim 1, further comprising:
a computer program product executable as a software application, resident on a mobile communication device in communication with the electronics unit, wherein the computer program product is able to control one or more of (i) detection of electrochemical measurements conducted at the microneedle array sensor unit, (ii) data analysis, (iii) data transmission, or (iv) device power management.

10. The device of claim 9, wherein the mobile communication device includes a smartphone, a smartwatch, a tablet, a smartglasses, a laptop, or one or more computers in the cloud.

11. The device of claim 1, wherein the plurality of analytes includes a diabetes biomarker including two or more of glucose, lactate, alcohol, or an electrolyte.

12. The device of claim 1, wherein one or more of the analytes are contained in a fluid deposited on the microneedle array sensor unit, the fluid including saliva, blood, tears, interstitial fluid, or combination thereof.

13. The device of claim 1, wherein the device can be configured as a patch worn on skin of a patient user.

14. (canceled)

15. A wearable medical device for collection of interstitial fluid (ISF), comprising:
a device body including a chamber enclosing an internal volume, the device body having a first side and a second side, wherein the first side of the device body is opposite to the second side of the device body;
an array of microneedles protruding from the first side of the device body, the array of microneedles operable to pierce skin when the first side of the device body is at or proximate to contact with the skin, wherein each microneedle in the array of microneedles comprises at least one exterior wall forming a protruding needle structure converging at an apex point, each microneedle having a hollow interior region defined by an interior wall that leads to an exterior opening located on the at least one exterior wall of the protruding needle structure, and wherein the hollow interior region of each microneedle in the array of microneedles is fluidically coupled to the internal volume of the device body through a first opening on the first side of the device body; and
a suction assembly disposed at the second side of the device body and fluidically coupled to the internal volume of the chamber through a second opening in the second side.

16. The device of claim 15 wherein the suction assembly comprises a fluidic channel capable of suction via capillary force, or a pump, or both the fluidic channel and the pump, fluidically coupled to the internal volume of the device body through the second opening on the second side of the device body.

17. The device of claim 16, wherein the second side of the device body comprises a pump port having a first end, a second end, a side surface enclosing a hollow internal volume of the pump port between the first end and the second end and having a first pump port opening into the internal volume of the pump port at the first end and a second pump port opening into the internal volume of the pump port at the second end, wherein the pump port is connected to the second side of the device body at the first end of the pump port such that the internal volume of the pump port is fluidically coupled to the internal volume of the device body through the first pump port opening into the internal volume of the pump port at the first end and the second opening on the second side of the device body.

18. The device of claim 16, wherein the pump is connected to the pump port using a tubing connected to the second end of the pump port.

19. The device of claim 15, wherein the suction assembly comprises
a bellow having a first bellow end, a second bellow end, a side surface enclosing a hollow internal volume of the bellow between the first bellow end and the second bellow end and having a first bellow opening into the internal volume of the bellow at the first bellow end;
a spring having a first spring end and a second spring end and positioned inside the bellow such that the first spring end is proximate to the first bellow end and the second spring end is proximate to the second bellow end, the spring is coiled around an axis connecting the first bellow end and the second bellow end, the spring contacts the device body at the first spring end, the spring contacts the bellow at the second spring end, and the spring is configured to contract and/or expand along the axis connecting the first bellow end and the second bellow end,
wherein:
the bellow is attached to the second side of the device body at the first bellow end such that the internal volume of the bellow is fluidically coupled to the internal volume of the device body through the first bellow opening into the internal volume of the bellow at the first bellow end and the second opening on the second side of the device body; and
the bellow is configured to transition between a contracted state and an expanded state wherein a distance between the first bellow end and the second bellow end in the expanded state is larger than the distance between the first bellow end and the second bellow end in the contracted state and wherein a transition of the bellow between the contracted state and the expanded state leads to an increase of the internal volume of the bellow.

20. The device of claim 15, wherein the array of microneedles includes a microneedle array sensor unit, comprising:
an array of micropillar electrodes, each disposed within the hollow interior region of a respective protruding needle structure of the array of microneedles, wherein a micropillar electrode of the array of micropillar electrodes is configured to interact with an analyte that comes in contact with a sensing region of the micropillar electrode via the exterior opening of the microneedle to produce an electrical signal indicative of an electrochemical reaction involving the analyte.

21. The device of claim 20, wherein the array of micropillar electrodes include a first electrode of the array configured to interact with a first analyte that interacts with the first electrode to produce a first electrical signal indicative of an electrochemical reaction involving the first analyte, and wherein a second electrode of the array is configured to interact with a second analyte that interacts with the second electrode to produce a second electrical signal indicative of an electrochemical reaction involving the second analyte.

22. The device of claim 20, wherein the device is configured to include or interface with an electronics unit to be in electrical communication with the array of micropillar electrodes, the electronics unit comprising a power source, a signal processing unit, and a wireless transmitter, wherein the device includes a plurality of surface-mount, spring-loaded pins that electrically couple the micropillar electrodes of the array to the signal processing circuit of the electronics unit.

23. A wearable microneedle sensor device for continuous analyte monitoring, comprising:
an array of microneedles, each comprising an exterior wall forming a protruding needle structure converging at an apex point, wherein each microneedle includes a hollow interior defined by an interior wall, and an opening disposed on the exterior wall leading to the hollow interior;
an array of micropillar electrodes, each disposed within the hollow interior of a respective protruding needle structure, wherein the micropillar electrodes of the array are configured to interact with an analyte that comes in contact with a sensing region of a respective micropillar electrode to produce an electrical signal indicative of an electrochemical reaction involving the analyte at the sensing region;
an electronics unit in electrical communication with the array of micropillar electrodes, the electronics unit comprising a power source, a signal processing circuit, and a wireless transmitter;
a plurality of surface-mount, spring-loaded pins that electrically couple the micropillar electrodes of the array to the signal processing circuit of the electronics unit; and
a housing structure to encase the electronics unit and to encase, at least partially, the array of microneedles and array of micropillar electrodes, such that at least the apex point and the opening of each microneedle of the array of microneedles protrude outward of the housing structure, wherein the array of microneedles is exposed from a side of the housing structure.

24. The device of claim 23, wherein the electronics unit includes a two-sided printed circuit board (PCB) to integrate electronic components including at least the signal amplification circuit, the wireless transmitter, and the power source, wherein a one side of the two-sided PCB includes the plurality of surface-mount, spring-loaded pins that electrically couple the micropillar electrodes to the signal amplification circuit and/or the power supply.

25. The device of claim 24, wherein the device includes an automatic power switch comprising at least one of the surface-mount, spring-loaded pins, wherein the automatic power switch is configured to provide an electrical conduction path through the array of microneedles in an on state when the array of micropillar electrodes are inserted within the array of microneedles, and wherein the electrical conduction path is broken when the micropillar electrodes are not inserted within the array of microneedles such that the automatic power switch is in an off state.

26. A wearable microneedle sensor device for continuous analyte monitoring, comprising:
a flexible, electrically-insulative substrate;
an array of microneedles disposed at a first region of the substrate, each microneedle of the array comprising an exterior wall forming a protruding needle structure converging at an apex point, wherein each microneedle includes a hollow interior defined by an interior wall, and an opening disposed on the exterior wall leading to the hollow interior;
an array of electrodes disposed at a second region of the substrate, wherein the second region includes a cavity having a bottom surface on which the electrodes of the array are positioned, wherein the electrodes includes a working electrode and at least one of a reference electrode or counter electrode, and wherein the array of electrodes are configured to detect an analyte in a fluid that is provided within the cavity such that the electrodes are operable to produce an electrical signal indicative of an electrochemical reaction involving the analyte at the working electrode;
a plurality of microfluidic channels disposed in the substrate between the cavity and the hollow interior of each microneedle of the array of microneedles, respectively;
an array of electrode interface terminals that are coupled to the array of electrodes, respectively, wherein each of the array of electrode interface terminals includes a contact region positioned away from a region that is coupled to the electrode, respectively;
an electronics unit in electrical communication with the array of electrode interface terminals, the electronics unit comprising a power source, a signal processing circuit, and a wireless transmitter; and
a plurality of surface-mount, spring-loaded pins that electrically couple the contact region of each of the electrode interface terminals of the array to the signal processing circuit of the electronics unit.

27. The device of claim 26, further comprising:
a housing structure to encase the electronics unit and to encase, at least partially, the microneedle array sensor unit, such that at least the apex point and the opening of each microneedle of the array of microneedles protrude outward of the housing structure, wherein the array of microneedles is exposed from a side of the housing structure.

28. The device of claim 27, wherein the housing structure is configured as a band that is wearable on an appendage of a patient user, such that the apex point of the microneedle is disposed on an inside face of the band.

29. The device of claim 28, wherein the band includes a digital display on an outside face of the band.

30. The device of claim 26, wherein the fluid includes at least one of saliva, blood, tears, interstitial fluid, or combination thereof.
